(19) United States
(12) Patent Application Publication
(10) Pub. No.: US 2021/0228497 A1
(43) Pub. Date: Jul. 29, 2021

(54) New Medical Devices, Delivery Vehicles and Manufacturing Thereof

(71) Applicant: Remy Biosciences, Inc., Irvine, CA (US)

(72) Inventor: Ludwig Weimann, San Diego, CA (US)

(21) Appl. No.: 17/051,043

(22) PCT Filed: Apr. 27, 2019
(86) PCT No.: PCT/US2019/029544

§ 371 Date: Oct. 27, 2020

Related U.S. Application Data
(60) Provisional application No. 62/663,900, filed on Apr. 27, 2018.

(51) Int. Cl. A61K 9/70; A61K 31/352; A61K 31/05; A61K 47/22; A61K 47/12; A61K 47/20; A61K 47/10; A61K 47/06; A61F 13/02

(52) U.S. Cl. CPC A61K 9/7061; A61K 31/352; A61K 31/05; A61K 47/22; A61K 47/12; A61F 2013/0296; A61K 9/7046; A61K 47/10; A61K 47/06; A61K 9/7084; A61F 13/0276; A61K 47/20

(57) ABSTRACT

The disclosure provides new medical device and manufacturing methods as well as chemicals that enhance delivery of pharmaceuticals from a transdermal patch, where the patch may include a film, adhesive, emulsifier, tackifier, or hydrogel.

### FIELD OF THE DISCLOSURE

The disclosure relates to transdermal delivery patches, tablets, capsules, and pills, as well as to buccal patches and dermal patches, each of which can contain a formulation providing a pharmaceutical agent such as a drug or a nutraceutical. The drug can be one or more cannabinoids.

### BACKGROUND OF THE DISCLOSURE

Dermal patches can take the form of a monolithic-style patch or a reservoir-style patch (see, U.S. LETTER U.S. Pat. No. 9,562,340 & 10,272,125 of Weimann). Monolithic-style patch can take the form of a sandwich, where the face that is exposed to the atmosphere is a backing, where the opposite face is a release liner, and where the filling of the sandwich is a matrix that includes an adhesive and a pharmaceutical agent such as a drug or nutraceutical. Prior to applying the patch to the skin, a release liner is removed and discarded.

Regarding reservoir-style patch, the reservoir can contain a pharmaceutical agent that is a drug or a nutraceutical. The reservoir also contains a liquid carrier and a gelling agent. The reservoir can be defined by a backing and by a permeable membraine, which together assume a “ravioli” conformation. The permeable membrane is optionally coated with an adhesive that mediates binding of the adhesive to the skin. On one side of the adhesive is the permeable membrane, and on the other side is a release linter. Prior to applying the patch to the skin, a release liner is removed and discarded.

Dermal patches are used to deliver capsaicin for reducing pain. The patch delivers capsaicin. Capsaicin acts on peripheral nociceptors. The patch can be applied for about one hour, where the result is pain reduction for many weeks (see, Peppin et al (2011) J. Pain Res. 4:385-392). Dermal patches are also used to deliver torigotine for treating Parkinson's disease, and where the patch provides continuous drug delivery over 24 hours, resulting in plasma pharmacokinetics similar to that with continuous i.v. infusions. Rotigotine acts on dopamine receptors (see, Elshoff et al (2015) Drugs. 75:487-501). To give another example, dermal patches can provide estrogen for therapy to post-menopausal women, and to provide ethinyl estradiol and norelgestromin for contraception. The contraceptive patch is used for 7 days, and it provides systemic concentrations similar to that with a daily oral contraceptive (see, Jung et al (2013) Drugs. 13:223-233).

The present disclosure provides sublingual tablets, capsules, pills, and strips, as well as buccal patches and dermal patches. These objects are provided herein as novel and enhanced tablets, capsules, strips, and patches that contain one or more drugs. Also provided are these same novel and enhanced objects, that do not contain one or more drugs, for example, as might find use as a placebo.

The present disclosure addresses an unmet need for sublingual tablets, capsules, and pills, dermal patches, sublingual patches, and buccal patches that provide pharmaceutical agents such as a cannabinoid, melatonin, capsaicin, lidocaine, salicylic acid, sildenafil, or a vitamin such as vitamin B1, vitamin D3, vitamin B12, or vitamin C.

### SUMMARY OF THE DISCLOSURE

Briefly stated, the present disclosure provides a composition capable of use in a buccal patches, sublingual patch, pill, tablet, or a dermal patch, wherein the composition comprises one or more of, an acrylic adhesive with non-functionality and an adhesive with only OH-functionality, further comprising one of more of enhancers selected from azone, oleic acid, and dimethylsulfoxide (DMSO); a polyisobutylene (PIB adhesive) with tackifiers that improve adhesion to skin using acrylic pressure sensitive adhesive mixed in at 1-50%, optionally with a cycloaliphatic hydrocarbon resin; a PIB adhesive with enhancers: at 3% of azone or oleic acid double the transdermal delivery from PIB; hemp oil with CBD of concentration 80-95% containing at least one terpene; a semisolid hydrogel that is saturated with cannabidiol (CBD) and tetrahydroxannabinol (THC); a semisolid hydrogel comprising an oil that consists essentially of CBD and THC (80-95%, wt/vol), in combination with ethanol/water (80/20, vol/vol), optionally with one or more enhancers selected from azone, oleic acid, and limonene; a semisolid hydrogel saturated with CBD and THC oils (80-95%, wt/vol), wherein the oil is mixed with EtOH/water (80/20, vol/vol), optionally with one or more enhancers selected from azone, oleic acid, and limonene; or a THC oil of THC (80-95%) mixed with 1-20% EtOH or with 1-10% EtOH/water (80/20, vol/vol) wherein including greater than 10% of ethanol is capable of lowering flux of THC delivery as determinable with a reservoir patch. Also provided is a buccal patch, sublingual pill, sublingual tablet, or sublingual patch, comprising one of the above compositions.

What is also embraced is a method for manufacturing the above patch, comprising the steps of combining THC, a film, an adhesive, and a backing, to generate an uncut patch, further comprising the uncut patch to produce a cut patch that is capable of applying to human skin or of applying to human buccal pouch.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual patent, and published patent application, as well as figures, drawings, sequence listings, compact discs, and the like, was specifically and individually indicated to be incorporated by reference.

Cannabinoids

The present disclosure provides dermal patches, formulations, dermal patches not containing a formulation, and dermal patches including a formulation. Preferred formulations include one or more cannabinoids. The major cannabinoids from Cannabis sativa are cannabidiol (CBD), cannabichromene (CBC), cannabigerol (CBG), delta-9-tetrahydrocannabinol (delta-9-THC), and cannabinol (CBN) (Appendino et al (2008) J. Nat. Prod. 71:1427-1430). Clinical trials have established that formulations derived from cannabis, can improve neuropathic pain of multiple sclerosis, improve appetite and sleep quality in cancer patients, relieve pain in fibromyalgia patients, and serve as an anti-emetic for chemotherapy induced nausea and vomiting (see, Health Canada (February 2013) Information for Health Care Professionals. Cannabis (Marihuana, Marijuana) and the Cannabinoids (152 pages)). The present disclosure also provides tetrahydrocannabinovarin (THCV), which is a propyl analogue of THC, and cannabidivarin (CBDV), which is a propyl analogue of CBD.

Formulations and compositions that include both THC and CBD at a given ratio are provided, such as at the ratio of about 95/5, about 90/10, about 80/20, about 70/30, about 60/40, about 50/50, about 40/60, about 30/70, about 20/80, about 10/90, and about 5/95 (by weight). Administering formulations containing both THC and CBD can have greater influence on reducing pain that formulations containing only THC or only placebo (see, Johnson et al (2010) J. Pain Symptom Management. 39:167-179; Notcutt et al (2004) Anaesthesia. 5944-452).

One of more of the following cannabinoids can be included in the compositions of the present disclosure. Cannaboids and related compounds further include, for example, cannabichromene; cannabitriol; cannabicyclolol; cannabielsoin, cannabinodiol; delta-8-tetrahydrocannabinol; cannabichromanone; cannabicoumaronone; cannabicitran; 10-oxo-delta-6a10a-tetrahydrocannabinol; cannabiglendol; delta-7-isotetrahydrocannabinol; CBLVA; CBV; CBEVA-B; CBCVA; delta-9-THCVA; CBDVA; CBGVA; divarinolic acid; quercetin; kaemferol; dihydrokaempferol; dihydroquercetin; cannflavin B; isovitexin; apigenin; naringenin; eriodictyol; luteolin; orientin; cytisoside; vitexin; canniprene; 3,4′-dihydroxy-5-methoxy bibenzyl; dihydroresveratrol; 3,4′-dihydroxy-5,3′-dimethoxy-5′-isoprenyl; cannabistilbene 1; cannabistilbene 11a; cannabistilbene 11b; cannithrene 1; cannithrene 2; cannabispirone; iso-cannabispirone; cannabispirenon-A; cannabispirenone-B; cannabispiradienone; alpha-cannabispiranol; beta-cannabispiranol; acetyl-cannabispirol; 7-hydroxy-5-methoxyindan-1-spiro-cyclohexane; 5-hydroxy-7-methoxyindan-1-spiro cyclohexane; myristic acid, palmitic acid, oleic acid, stearic acid, linoleic acid, linolenic acid, arachidic acid, eicosenoic acid, behenic acid, lignoceric acid, 5,7-dihydroxyindan-1-cyclohexane; cannabispiradienone; 3,4′-dihydroxy-5-methoxybibenzyl; canniprene; cannabispirone; cannithrene I; cannithrene 2; alpha-cannabispiranol; acetyl-cannabispirol; vomifoliol; dihydrovomifoliol; beta-ionone; dihydroactinidiolide; palustrine; palustridine; plus-cannabisativine; anhydrocannabisativine; dihydroperiphylline; cannabisin-A; cannabisin-B; cannabisin-C; cannabisin-D; grossamide; cannabisin-E; cannabisin-F; cannabisin-G; and so on (see, e.g., Flores-Sanchez and Verpoorte (2008) Secondary metabolism in cannabis. Phytochem. Rev. 7:615-639).

In exclusionary embodiments, the present disclosure can exclude any formulation, composition, device, or method that comprises CBD, CBC), CBG, delta-9-THC, CBN, or any chemical in the above list. What can be excluded is any formulation, composition, device, or method, that takes the form of a liquid cannabinoid formulation where at least 20%, at least 40%, at least 60%, at least 80%, at least 90%, or at least 95%, of total cannabinoids is tetrahydrocannabinolic acid (THCa). Also, what can be excluded is any formulation, composition, device, or method, that takes the form of an oil formulation, where the oil formulation contains one or more cannabinoids, where at least 20%, at least 40%, at least 60%, at least 80%, at least 90%, at least 95% of total cannabinoids is tetrahydrocannabinolic acid (THCa).

Measuring Cannabinoids

Cannabinoids can be separated, purified, analyzed, and quantified by a number of techniques. Available equipment and methods include, e.g., gas chromatography, HPLC (high pressure liquid chromatography, high performance liquid chromatography), mass spectrometry, time-of-flight mass spectrometry, gas chromatography-mass spectrometry (GC-MS), and liquid chromatography-mass spectrometry (LC-MS). Equipment for separation and analysis is available from Waters Corp., Milford, Mass.; Agilent, Foster City, Calif.; Applied Biosystems, Foster City, Calif.; and Bio-Rad Corp., Hercules, Calif. Methods, equipment, and manufacturers for HPLC fractionation and identification of cannabinoids are disclosed (see, e.g., Peschel W (2016) Quality control of traditional cannabis tinctures. Sci. Pharm. 84:567-584; Scheidweiler K B et al (2012) Simultaneous quantification of free and glucuronidated cannabinoids in human urine by liquidchromatography tandem mass spectrometry. Clin. Chim. Acta. 413:1839-1847).

The present disclosure provides in-line monitoring of purification, that is, quantitation of THC as well as quantitation of impurities. In-line monitoring may be by UPLC methods, or by other methods. Ultra-high performance liquid chromatography (UPLC) is similar to HPLC, except that UPLC uses smaller particles in the column bed, and greater pressures. The particles can be under 2 micrometers in diameter, and pressures can be nearly 15,000 psi. UPLC also uses higher flow rates, and can provide superior resolution and run times in the range of under 30 seconds (Wren and Tchelitcheff (2006) J. Chromatography A. 1119:140-146; Swartz, M. E. (May 2005) Separation Science Redefined). The application of UPLC to cannabinoids has been described (see, Jamey et al (2008) J. Analytical Toxicology. 32:349-354; Badawi et al (2009) Clinical Chemistry. 55:2004-2018). Suitable UPLC columns for cannabinoid analysis include, e.g., Acquity®UPLC HSS T3 C18, and Acquity® UPLC BEH C18 column (Waters, Milford, Mass.). Other methods for detecting cannabinoids include, e.g., infrared (IR) spectroscopy, gas chromatography mass spectroscopy (GCMS), and electrospray tandem mass spectroscopy (ESI-MS/MS) (Ernst et al (2012) Forensic Sci. Int. 222:216-222).

Biochemical properties of cannabinoids, binding to cannabinoid receptors, terpenes and terpene receptor binding, can be assessed using labeled cannabinoids, labeled terpenes, and labeled ligands where a cannabinoid or a terpene influences binding properties of the labeled ligand. Useful labels include radioactive labels, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

Cannabinoid Numbering Systems

The present disclosure uses the nomenclature as set forth by Pertwee R G et al (2010) International Union of Basic and Clinical Pharmacology. LXXIX. Cannabinoid receptors and their ligands: beyond CB1 and CB1. Pharmacol. Rev. 62:588-631. Regarding different numbering systems for the same compound, Aviv (US 2004/0110827) states that: “It should be noted that for historical reasons, these cannabinoid analogs are still named following the previous nomenclature, where the terpenic ring was the base for the numbering system. Then the chiral centers of THC type cannabinoids were at carbon atoms 3 and 4. The accepted nomenclature is now based on the phenolic ring as the starting point for numbering. Thus, THC that was previously described as delta-1-THC was later renamed delta-9-THC, similarly delta-6-THC was renamed delta-8-THC, and the chiral centers are at carbons 6a and 10a.” AVIV also has this comment about enantiomers: “delta-9-THC was established by Mechoulam R. et al. in 1967 and found to be of (−)-(3R,4R) stereochemistry. It was later found that the psychotropic activity of cannabinoids resides in the natural (3R,4R) OH series, while the opposite enantiomeric synthetic series (3S,4S) was free of these undesirable effects.”

According to Agurell (1988) Pharmacological Revs. 38:21-43, the terpene numbering system uses delta-1-THC, while the dibenzopyran system uses delta-9-THC to refer to the same chemical. Both of these numbering systems can be used for THC, CBD, and CBN.

According to Chulgin, the numbering system most broadly used recognizes both the terpene nature and the aromatic nature of the two different parts of the cannabinoid. Here, the terpene is numbered from the ringcarbon that carries that branched methyl group, and this is numbered 7, and the remaining three carbons of the isopropyl group are then numbered sequentially. The advantage to this numbering system is that this numbering system is applicable whether the center ring is closed or open. Other numbering systems are the biphenyl numbering system, the Chemical Abstracts system (substituted dibenzopyran numbering), and the Todd numbering system (pyran numbering) (see, Chulgin A T (1969) Recent developments in cannabis chemistry. J. Psychedelic Drugs. pp. 397-415.

Hemp Oil, Other Oils, and Extracts

Hemp oil or hempseed oil is obtained by pressing hemp seeds. It is an edible oil that contains about 80% of essential fatty acids but it is not CBD hemp oil.

Cannabidiol (CBD) hemp oil can be manufactured using Supercritical Carbon Dioxide (CO₂) extraction of the stalk of the industrial hemp plant. CBD hemp oil usually contain 20-40% CBD. To produce crystalline CBD of 99.8% pure, the CBD oil is processed further using fractional distillation.

Marijuana extract oils are extracts of marijuana plants such as Sativa or Indica. The extracts are solids or semisolids of different % of CBD, THC and other cannabinoids. Marijuana THC extracts (oils) may have high % of THC up to 80% and some CBD about 10-20%.

Marijuana CBD extracts may have high % of CBD up to 90% and low % of THC up to 10%. The percentage of cannabinoids in the marijuana extracts depend on the content of those substances in marijuana plants that are used in extraction. Extraction are done by using butane, ethyl alcohol or critical CO₂ extraction.

Hemp oil suppliers are listed here (see, e.g., Medical Marijuana, Inc., Poway, Calif.; Nutiva, Richmond, Calif.; Entourage Nutritional Distributors, Colorado Springs, Calif.). Hemp ground in Poland, for example, has been described (see, e.g., L. Grabowska et al (2009) Breeding and cultivation of industrial hemp in Poland. Herba Polonica. 55:328-334; L. Grabowska et al (2008) Maintenance breeding of Polish hemp cultivar Beniko. J. Natural Fibers. 5:208-217). Varieties (cultivars) of hemp grown in Poland and adapted to Polish climate and soil conditions include, Bialobrzeslcie, Beniko, Silesia, Tygra, and Wielkopolskie.

Since CBD hemp oil can be produced without stripping it from terpenes, the transdermal formulation of the present disclosure provides information if the natural terpenes facilitate the transdermal skin penetration of CBD and information on how the natural terpenes provide medicinal properties once absorbed through the skin.

### Matrix Embodiments

An excipient useful for granulating agents and sprays is the polyvinylpyrrolidone copolymer having a given ratio, or range of ratios, of polyvinylpyrrolidone/vinyl acetate (PVP/VA). The present disclosure provides PVP/VA (or combinations of any two polymers), at a ratio of 10/90, 20/80, 30/70, 40/60, 50/50, 60/40, 70/30, 80/20, 90/10, as well as a combination of any two polymer at a ratio of about 10/90, about 20/80, about 30/70, about 40/60, about 50/50, about 60/40, about 70/30, about 80/20, about 90/10. Also, the present disclosure can exclude PVP/VA compositions (or it can exclude a combination of any two polymers) with a ratio of, 10/90, 20/80, 30/70, 40/60, 50/50, 60/40, 70/30, 80/20, 90/10, or about 10/90, about 20/80, about 30/70, about 40/60, about 50/50, about 60/40, about 70/30, about 80/20, about 90/10, and the like. The PVP/VA copolymer has the ability to distribute homogeneously around an active ingredient during formation of an aqueous liquid phase (see, US2016/0058866 of Sekura). Polymers and copolymers are available from Sigma-Aldrich, St. Louis, Mo., Nippon Shokubai Co., Ltd., Osaka, Japan, BASF Corp., Florham Park, N.J., and Ashland, Schaffhausen, Switzerland.

In methods of manufacturing embodiments, monolith patch can be made as follows. Cannabis oil or one or more pure cannabinoids can be combined with permeation enhancer only, combined with carrier only, or combined with both permeation enhancer and carrier. Carrier can comprise, for example, one or more of oleic acid and dodecylmethyl sulfoxide. Then one or more pure terpenes, or an essential oil, or a combination of an essential oil and one or more pure terpenes, is mixed with the above combination. Then, a polymer such as a silicone polymer is mixed in. Finally, the mixture is spread into one or more sheets, cured at room temperature for several hours or longer. After drying, a foam backing layer is applied, and then the product is cut into shapes (e.g., squares, rectangles, ovals, round-edged squares or round-edged rectangles, circles) suitable for applying to the skin of a person.

A laminate that can be held in place on the gingiva (gums) takes the form of a semipermeable outer layer, reservoir having a pharmaceutical, backing layer, where the backing layer faces the gingiva. Saliva can enter through the semipermeable outer layer, pass through the reservoir, and then draw medicine into contact with gingiva for absorption in the bloodstream. A pharmaceutical can be freeze dried or can occur as a hydrogel matrix, in the reservoir. The present disclosure provides a backing layer of one or more polymers, such as, ethyl cellulose, butyl cellulose, hydroxybutyl cellulose, or polyvinylalcohol. An amorphous or semi-crystalline excipient matrix can be made from methylcellulose, ethylcellulose, hydroxypropyl methylcellulose, cellulose acetate phthalate, or cellulose acetate butyrate. In exclusionary embodiments, the present disclosure can exclude one or more of these polymers.

In reservoir-distribution embodiments, a pharmaceutical or nutraceutical can be distributed evenly throughout reservoir, or can be distributed at a higher concentration at center of reservoir, or can be distributed at a higher concentration at region of reservoir that is closer to the skin when patch is situated and adhering to skin.

Tackifiers

The present disclosure provides compositions, patches, and methods, that encompass one or more of Escorez 1000 Series-aliphatic resins; Escorez 2000 Series-aromatic modified aliphatic resins; Escorez 5300 Series-water white hydrogenated cycloaliphatic resins; Escorez 5400 Series-light color hydrogenated cycloaliphatic resins; Escorez 5600 Series-light color hydrogenated aromatic modified cycloaliphatic resins; Escorene® Ultra ethylene vinyl acetate (EVA) copolymers; ExxonMobil® ethylene n-butyl acrylate (EnBA) copolymers; Optema® EMA (ethyl methyl acrylate) resins (ExxonMobil, Inc.).

Escorez® 5400 is a hydrocarbon polymer additive available from ExxonMobil Chemical Company. It has a softening point of 103° C., a weight average molecular weight of about 400 g/mole, and a dicyclopentadiene/cyclopentadiene/methylcyclopentadiene content of 40-80 wt % (see, WO2013/176712 of Block).

Escorez® 5415 is a hydrocarbon polymer additive available from ExxonMobil Chemical Company. It has a softening point of 118° C., a weight average molecular weight of about 430 g/mole, and a dicyclopentadiene/cyclopentadiene/methylcyclopentadiene content of 40-80 wt % (see, WO2013/176712 of Block).

Escorez® 5340 is a hydrocarbon polymer additive available from ExxonMobil Chemical Company. It has a softening point of 140° C., a weight average molecular weight of about 460 g/mole, and a dicyclopentadiene/cyclopentadiene/methylcyclopentadiene content of 40-80 wt % (see, WO2013/176712 of Block).

Escorez® 5600 is a hydrocarbon polymer additive available from ExxonMobil Chemical Company. It has a softening point of 103° C., a weight average molecular weight of about 520 g/mole, and a dicyclopentadiene/cyclopentadiene/methylcyclopentadiene content of 40-80 wt % (see, WO2013/176712 of Block).

Escorez® 5615 is a hydrocarbon polymer additive available from ExxonMobil Chemical Company. It has a softening point of 118° C., a weight average molecular weight of about 500 g/mole, and a dicyclopentadiene/cyclopentadiene/methylcyclopentadiene content of 40-80 wt % (see, WO2013/176712 of Block).

Hydrogels

Hydrogels are 3-dimensional, cross-linked networks of water-soluble polymers. The porous structure of hydrogels can be altered by changing the density of cross-linking. The degree of cross-linking can alter the rate of loading a drug, and it can alter the rate of drug release. The present disclosure can encompass a hydrogel that consists of one of the following polymers or alternatively, that comprises one or more of the following polymers (e.g., as a block polymer). The polymers include, poly(ethylene oxide) (PEO), poly(propylene oxide) (PPO), poly(lactide-co-glycolic acid) (PLGA), poly(N-isopropylacrylamide) (PNIPAM), poly(propylene fumarate) (PPF), poly(caprolactone) (PCL), poly(urethane) (PU), and poly(organophosphazene) (POP). An example of a block polymer is PEO-PPO-PEO. In exclusionary embodiments, the present disclosure can exclude a hydrogel that includes PEO, PPO, PLGA, PNIPAM, PPF, PCL, PU or POP. The present disclosure also encompasses hydrogels that contain a cyclodextrin, where the cyclodextrin is cross-linked to hydrogel (see, Hoare et al (2008) Hydrogels in drug delivery: Progress and challenges. Polymer. 49:1993-2007). Hydrogels of the present disclosure can be ethylene vinylacetate, alginic acid, gums, polyvinylalcohol hydrogel; silicone hydrogel; polyvinylalcohol/dextran hydrogel; alginate hydrogel; alginate-pyrrole hydrogel; gelatin/chitosan hydrogel; polyacrylic acid hydrogel; photo crosslinked polyacrylic acid hydrogel; amidated pectin hydrogel; pectin hydrogel; gelatin hydrogel; polyethylene glycol (PEG) hydrogel; carboxymethylcellulose/gelatin hydrogel; chitosan hydrogel, as well as mixtures thereof, or copolymers thereof, and the like. Hydrogel with crosslinks are available (Lee et al (2003) Eur. J. Pharm. Biopharm. 56:407-412).

Printing Active Ingredients and Excipients on Dried Hydrogels

Dried hydrogel can take the form of a “xerogel” or of a film. Xerogel can be made by freeze drying a hydrogel. Film can be made by evaporative drying or casting from organic solutions. Spotting device can be used to apply microdrops in predetermined locations of dried hydrogel or on a film (see, e.g., U.S. Pat. No. 6,642,054 of Schermer). Where dried hydrogel or film takes the form of a layer, microdrops can be applied to one side only or to both sides. Where more than one type of drug is to be applied and where at least two of the drugs are incompatibile with each other, or where a drug and an excipient are to be applied, and where these are incompatible with each other, these can be applied at different locations on the dried hydrogel or on the film. Drop size of microdrops can be, e.g., 0.05 nanoliters (nL)-10,000 nL, 0.5 nL-200 nL, 10 nL-100 nL, and so on. Drug, active ingredient, and/or excpient is not incorporated into the dried hydrogel, but is instead printed on its surface or surfaces. Printing on dried hydrogel avoids problems arising from incompatibiliy of drug, active ingredient, and/or excipient with the hydrogel itself. See, US2008/0095848 of Stabenau, which is incorporated by reference in its entirety.

Cyclodextrins

Cyclodextrins are cyclic oligosaccharides of (alpha-1,4)-linked alpha-D-glucopyranose units, with a lipophilic central cavity and a hydrophilic outer surface. As a result of their molecular structure and shape, they can act as molecular containers by trapping drugs or other molecules in their internal cavity. No covalent bonds are formed or broken during drug cyclodextrin complex formation, and in aqueous solution, the complexes readily dissociate and free drug molecules remain in equilibrium with the molecules bound within the cyclodextrin cavity (see, Tiwari et al (2010) Cyclodextrins in delivery systems: Applications. J. Pharm. Bioallied Sci. 2:72-79). Derivatives of cyclodextrins that are hydroxypropyl (HP), methyl (M) and sulfobutylether (SBE) substituents are useful as pharmaceutical excipients.

Cyclodextrins for use, for example, in cannabinoid/cyclodextrin complex, include beta-cyclodextrins such as hydroxypropyl-beta-cyclodextrin, sulfobutylether-beta-cyclodextrin, maltoxyl-beta-cyclodextrin, and methylated cyclodextrins. Encompassed are alpha-cyclodextrins (6 glucopyranose units), beta-cyclodextrins (7 glucopyranose units), and gamma-cyclodextrins (8 glucopyranose units). Methylated cyclodextrins can improve acqueous solubility, dissolution rate, and bioavailability of cannabinoids.

The present disclosure provides a dermal patch (or buccal patch) comprising a dextrin where the dextrin is not complexed with a pharmaceutical agent, and a dermal patch (or buccal patch) comprising a dextrin where the dextrin is, in fact, complexed with a pharmaceutical agent.

In exclusionary embodiments, the present disclosure can exclude a formulation that comprises a cyclodextrin, or that comprises an alpha-cyclodextrin, or that comprises a beta-cyclodextrin, or that comprises a gamma-cyclodextrin. What can also be excluded is a device that comprises a cyclodextrin, such as an adhesive dermal patch comprising a dextrin or a buccal patch comprising a dextrin.

Matrices, Carriers, Binders, Tablets, Pills, Manufacturing Methods

A matrix, carrier, or binder, can include, e.g., hydrogel, polyethylene oxide, polyvinylpyrrolidone, hydroxypropyl cellulose, ethyl cellulose, methylcellulthose, alkylcelluloses, veegums clays, alginates, PVP, alginic acid, carboxymethylcellulose calcium, microcrystalline cellulose, polacrillin potassium, sodium alginate, corn starch, potato starch, pregelatinized starch, corn starch, modified starch, carnuba wax, montmorrilonite clays such as bentonite, gums, shellac, agar, locust bean gum, gum karaya, pecitin, tragacanth, and the like. In exclusionary embodiments, what can be excluded is one or more of the above polymers, clays, waxes, hydrogels, starches, and gums. A polyol can be used, for example, as a carrier. Polyols include propylene glycol and glycerol and the preferred (poly) alkoxy derivatives include polyalkoxy alcohols, in particular 2-(2-ethoxyethoxy) ethanol (Transcutol®).

Gums suitable for buccal tablets are disclosed in U.S. Pat. No. 4,829,056, which is incorporated by reference in its entirety. Lozenges and sublingual pills are provided, and these can comprise one or more of sodium phosphate, potassium phosphate, guar gum, gum arabic, locust bean gum, xanthan gum, carrageenan, carob gum, ghatti gum, pectin, tragacanth gum, acacia gum, mannitol, sorbitol, lactose, modified lactose, maltitol, mannitol, magnesium stearate, hydroxypropylmethylcellulose film, non-crystallizing sugar, or non-crystallizing sugar alcohol.

Matrix can be manufactured by melt-granulation, melt-extrusion, using particulates, granules, bilayers, plasticizers, and the like (see, US2016/0151502 of Wright). Patch can be made with silicone adhesives disposed on a substrate, copolymers, block polymers, tackifying resins, hot melt coating processes (see, US2014/0349108 of Fung). Patch can be made with backings, release liner, pressure sensitive adhesives, silicone gel adhesives (see, US2014/0287642 of Kumar). Dermal patch, buccal patch, tablets, can be made with excipient, disintegrant, swelling agent, films, binders, and the like (US2014/0079740 of Salama). Each of these patent documents is incorporated herein by reference in its entirety. Hot-melt extrusion, granules, tablets, transmucosal patches, transdermal patches, and methods of manufature are detailed (Crowley et al (2007) Drug Development Industrial Pharmacy. 33:909-926; Repka et al (2007) Drug Development Industrial Pharmacy. 33:1043-1057).

Regarding sublingual tablets, sublingual pills, and sublingual strips, equipment for compressing granules, for applying coatings and lubricants, are available (see, US2010/0233257 of Herry). Regarding sublingual tablets and buccal tablets, formulas involving, e.g., cross-linked carboxymethylcellulose, lactose, microcrystalline cellulose, binding liquids, and equipment such as drier, mixer-granulator, compressor, are disclosed (see, e.g., U.S. Pat. No. 9,308,212). Penetration enhancers, fillers, binders, carriers, equipment for molding and solidifying sublingual tablets are disclosed (U.S. Pat. No. 9,220,747 of Gould). Each of these patent documents is incorporated herein by reference in its entirety.

Apertures and Pores

The present disclosure can encompass films, sheets, layers, membranes, and the like, including those with a plurality of apertures or pores. In some aspects, the apertures or pores have an average diameter of 20 nm, 40 nm, 50 nm, 100 nm, 200 nm, 300 nm, 400 nm, 500 nm, 600 nm, 800 nm, 0.001 mm, 0.002, 0.005 mm, 0.010 mm, 0.015 mm, 0.020 mm, 0.025 mm, 0.030 mm, 0.040 mm, 0.050 mm, 0.075 mm, 0.10 mm, 0.20 mm, 0.30 mm, 0.40 mm, 0.50 mm, and the like. Also, the pores can have a diameter range where the range is bracketed by any two of these values. In other aspects, the apertures or pores have a diameter in the range of 20-40 nm, 40-60 nm, 60-80 nm, 50-100 nm, 100-200 nm, 200-400 nm, 400-600 nm, 600-800 nm, 800-1,000 nm, 0.001-0.002 mm, 0.001-0.005 mm, 0.005-0.010 mm, 0.010-0.020 mm, 0.020-0.040 mm, 0.025-0.050 mm, 0.050-0.075 mm, 0.075-0.10 mm, 0.10-0.20 mm, 0.20 mm-0.40 mm, 0.25-0.50 mm, 0.50-0.75 mm, 0.50-1.00 mm, 0.1-0.2 mm, and so on. In exclusionary embodiments, the present disclosure can exclude films, sheets, layers, and the like, that have apertures or pores having any of the above average values, or that are describable by any of the above ranges.

Porous membranes can take the form of hydrophilic porous membranes and hydrophobic porous membranes, without implying any limitation. Hydrophobic membranes, such as hydrophobic polyehtylene (PE) membranes, can be made more hydrophilic by alcohol or surfactants (see, WO2010/072233 of Calis). Pores in membranes of the present disclosure can have an average diameter of about 5 micrometers, about 10, about 15, about 20, about 25, about 30, about 40, about 50, about 60, about 70, about 80, about 90, about 100, about 110, about 120, about 130, about 140, about 150, about 160, about 170, about 180, about 190, or about 200 micrometers, and the like. Also, pores in the membranes can have an average diameter somewhere in the range 5-20 micrometers, 20-40 micrometers, 40-60 micrometers, 60-80 micrometers, 80-100 micrometers, 100-120 micrometers, 120-140 micrometers, 140-160 micrometers, 160-180 micrometers, 180-200 micrometers, and so on. In exclusionary embodiments, the present disclosure can exclude any membrane that is characterized by one of the above “about” values or that is characterizable by one of the above ranges.

For any given film, sheet, or layer, and the like, the area of a plurality of apertures or the area of a plurality of pores can occupy about 1%, about 2%, about 4%, about 6%, about 8%, about 10%, about 15%, about 20%, about 25%, about 30%, about 35%, about 40%, about 45%, about 50%, about 55%, about 60%, about 65%, about 70%, about 75%, about 80%, about 85%, about 90%, and the like of the surface area. In exclusionary embodiments, the present disclosure can exclude any film, sheet, or layer, where the area does not occupy one or more of the given percentage values, or where the area does not occupy a range between any two of the above given percentage values. The above parameters also can apply to a film, sheet, or layer, with perforations, where the value of the area for the perforation is measured flush with a surface of the film, sheet, or layer.

Solubilizers and Surfactants

Solubilizers such as detergents, surfactants, organic solvents, and chaotropic agents, are available for the present disclosure. These can be one or more of, polyethylene glycol (PEG), propylene glycol, dibutyl subacetate, glycerol, diethyl phthalate (phthalate esters), triacetin, citrate esters-triethyl citrate, acetyltriethyl citrate, tributyl citrate, acetyltributyl citrate, benzyl benzoate, sorbitol, xylitol, bis(2-ethyllhexyl) adipate, mineral oil, polyhydric alcohols such as glycerin and sorbitol, glycerol esters such as glycerol, triacetate; fatty acid triglycerides, polyoxyethylene sorbitan, fatty acid esters such as TWEENS, polyoxyethylene monoalkyl ethers such as BRIJ series and MYRJ series, sucrose monoesters, lanolin esters, lanolin ethers. These are available from Sigma-Aldrich, St. Louis, Mo. In exclusionary embodiments, what can be excluded is any composition, formulation, dermal patch, and methods that comprise one or more of these solubilizers or surfactants.

The present disclosure can encompass compositions, formulations, devices, and methods, that comprise one or more surfactants, such as, sorbitan trioleate, sorbitan mono-oleate, sorbitan monolaurate, polyoxyethylene (20) sorbitan monolaurate, polyoxyethylene (20) sorbitan monooleate, oleyl polyoxytheylene (2) ether, stearyl polyoxyethylene (2) ether, lauryl polyoxyethylene (4) ether, block copolymers of oxyethylene and oxypropylene, diethylene glycol dioleate, tetrahydrofurfuryl oleate, ethyl oleate, isopropyl myristate, isopropyl palmitate, glyceryl monooleate, glyceryl monostearate, glyceryl monoricinoleate, cetyl alcohol, stearyl alcohol, cetyl pyridinium chloride, olive oil, glyceryl monolaurate, corn oil, cotton seed oil, and sunflower seed oil. In exclusionary embodiments, the present disclosure can exclude one or more of the above chemicals, and can also exclude a composition, formulations, device, and method that comprises any of the above chemicals.

Buffers and pH Values

The present disclosure can include formulations that contain a buffer with a pKa, as measured at room temperature, such as boric acid (pKa 9.2), CHES (pKa 9.5), bicine (pKa 8.3), HEPES (pKa 7.5), MES (pKa 6.1), MOPS (pKa 7.2), PIPES (pKa 6.8), Tris (pKa 8.1), imidazole (pKa 6.9), glycine (pKa 2.3), acetate (pKa 4.7), citrate (pKa 6.4), phosphate (pKa 7.21, 2.16, 12.32), malate (pKa 5.13), cacodylate (pKa 6.27), and the like. Also, the present disclosure can exclude formulations that include one or more of the above buffers, and can exclude a device that comprises one of these formulations. Without regard to any buffer, the present disclosure provides a formulation, or provides a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. In exclusionary embodiments, the present disclosure can exclude a formulation, or can exclude a component of a formulation, that has a pH value, as measurable at room temperature, of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, and the like. The pH of component can be measured as pure component, that is, prior to combining with other components to generate formulation.

Buccal Patches, Sublingual Patches, and Related Pills, Tablets, and Strips

The present disclosure encompasses patch-based delivery systems for use in the mouth. In the mouth, regions for drug delivery include sublingual mucosa (area beneath the tongue) and the buccal mucosa (inner lining of the cheeks). Buccal administration of low water-solubility pharmaceuticals can be enhanced by formulating pharmaceutical in combination with a surfactant, or as a complex with hydrophilic cyclodestrins, or by using a nanosuspension (particle diameter in the nanomolar range, such as 50 nm to 150 nm) (see, Rao et al (2011) Int. J. Nanomedicine. 6:1245-1251). Nanoparticles can be made by milling, homogenization, or ultrasonication.

Buccal pouch is space between the cheek and the gums. Buccal dosage forms are inserted into the buccal pouch (see, U.S. Pat. No. 8,735,374 of Zerbe, which is incorporated herein in its entirety). Buccal patch can include an emulsifier that, when exposed to water, results in hydration-induced formation of an emulsifier. Emulsion can form spontaneously, that is, without much energy supply or without shearing forces, when water contacts the emulsifier. When placed against the gums, saliva drawn into the buccal patch can be the source of water. Self-emulsifying agent enhances the tendency of the formulation to adhere to the mucosal surface, thus promoting absorption of pharmaceuticals such as cannabinoids (see, U.S. Pat. No. 7,709,536 of Dam and U.S. Pat. No. 8,642,080 of Bender, each of which is incorporated herein by reference in its entirety).

This describes solvent casting and direct milling methods of manufacture. Without implying any limitation, buccal patch can consist of two laminates, with an aqueous solution of an adhesive polymer being cast on an impermeable backing sheet. One type of adhesive film can comprise an alcoholic solution of hydroxypropyl cellulose and organic acids. This adhesive film stays in place for at least 12 hours, even in the presence of fluids. Adhesive patches can be made by solvent casting or by direct milling. In solvent casting, all excipients and the drug are dispersed in an organic solvent and coated on a sheet of release liner. After solvent evaporates, a thin layer of protective material is laminated on the sheet of coated release liner to form a laminate. The laminate is then cut into patches (Koyi and Khan (2015) Buccal patches: A review. Int. J. Pharmaceutical Sciences Res. 4:83-89).

In direct milling, patches are created without using solvents. Drug and excipients are mixed by direct milling or by kneading, usually without any liquids present. After milling, the material is rolled on a release liner. A backing layer is then applied. Direct milling avoids the problem of residual solvents (Koyi and Khan (2015) Buccal patches: A review. Int. J. Pharmaceutical Sciences Res. 4:83-89).

The concerns solvent casting method and hot melt extrusion method. Without implying any limitation, buccal film can be made by solvent casting method and by hot melt extrusion method. Solvent casting involves dissolving water-soluble polymers to form viscous solution. Excipients are dissolved into solvent to give clear viscous solution. Then, both solutions are mixed (solution of water-soluble polymers; excipient solution) and then cast as a film, and then allowed to dry. This concerns hot melt extrusion. The drug or combination of drugs is in a dry state, and it is filled in a hopper, mixed, heated, and then extruded in a molten state. The molten mass that is formed is used to cast a film (Madhavi et al (2013) Buccal film drug delivery system—an innovative and emerging technology. J. Mol. Pharm. Org. Processing Res. Vol. 1, Issue 3 (6 pages)).

Without implying any limitation, mucoadhesive patches can be made by dissolving polymers in a solvent to produce a viscous solution. The polymers can be hydroxypropylmethyl cellulose (HPMC) E5LV and Carbopol® 940P. Polyethylene glycol 1000 can be included as a plasticizer. The solvent can be ethanol:chloroform (50:50). After creating of the viscous solution, drug can be dispersed in it. Then, the solution can be poured into molds for casting and dried for 24 hours. After drying, patches can be cut, for example, at 2 cm×2 cm. Each of the patches can contain, for example, 2 mg drug, 20 mg HPMC, 0.4 mg Carbopol, and 17 mg PEG100 (wt/vol) (see, Priya et al (2011) J. Pharm. Res. 3:56-65).

Franz Diffusion Cell for Assessing Drug Release

Franz diffusion cell is used to measure drug release kinetics from monolithic patches and from reservoir patches. Franz diffusion cells are described (see, Cavallari et al (2013) Eur. J. Pharm. Biopharm. 83:405-414; Franz (1968) On the diffusion of tritiated water through skin. J. Invest. Dermatol. 50:260; Balazs, Sipos, Danciu (2015) Biomedical Optics Express. 7:67-78; Simon et al (2016) Int. J. Pharmaceutics. 512:234-241; Jung et al (2016) Int. J. Cosmet. Sci. 38:646-650; Technical Brief 2009, Vol. 10, Development and Validation In Vitro Release Testing Methods for Semisolid Formulations, Particle Sciences, Bethlehem, Pa.). Franz diffusion cells and equipment for transdermal diffusion testing are available (Teledyne Hanson Research, Chatsworth, La.).

The present inventor used a Franz cell for assessing release kinetics, as described below. For testing release kinetics from transdermal monolithic patches, where a semi-solid adhesive matrix is used, the Franz cell has the following components, from top to bottom: (1) Stopper used to seal top of donor compartment; (2) Donor compartment; (3) Patch situated at very bottom of donor compartment, with adhesive side of patch attached to human cadaver skin; (4) Human cadaver skin located immediately under the patch; (5) Receiving compartment, located immediately below the skin. Receiving compartment is filled with ethanol/water solution; (6) Magnetic stirrer located at bottom of receiving compartment.

For testing release kinetics from transdermal reservoir patches, the Franz cell has the following components, from top to bottom: (1) Stopper used to seal top of donor compartment; (2) Donor compartment; (3) Cream or gel in the donor compartment; (4) Microporous membrane (Solupor® from Lydall Performance Materials, Inc., Rochester, N.H.); (5) Human cadaver skin located under the microporous membrane; (6) Receiving compartment is filled with phosphate buffered saline at pH 6; (7) Magnetic stirrer at bottom of receiving compartment.

Receiving Solutions for the Franz Cell Used to Test Patches of the Present Disclosure

This concerns receiving solutions that were used for testing the monolithic patches and dermal patches of the present disclosure. The receiving solution can be a saline solution for drugs that are soluble in water or alcohol-water solutions for drugs that are not well soluble in saline.

According to Bartosova and Bajgar (2012) Current Medicinal Chemistry. 19:4671-4677, demal absorption involves these steps: (1) Penetration. Entry of a substance into a particular layer of the skin, such as the layer that is “stratum corneum;” (2) Permeation. This is penetration through one layer into another slayer, where the layers differ both structurally and functionally from each other; (3) Absporption. Uptake of substance into the lymphatics or into the bloodstream. The stratum corneum is lipophilic while, in contrast, the epidermis and dermis are hydrophilic. Thus, lipophilicmolecules may pass at a greater rate through stratum corneum while, in contrast, hydrophilic molecules may pass at a greater rate through epidermis and dermis. Rate of transfer can be expressed by Frick's law: Jₛₛ=(Kₚ)(Cₒ). Jₛₛ is steady state flux per unit area. Kₚ is permeability coefficient for a given solute in a given vehicle (centimeters per hour). Cₒ is concentration of solute in the donor compartment. Kₚ predicts the penetration rate of a chemical at a given concentration from the same vehicle. Kₚ is independent of conctration and time.

According to Bartosova and Bajgar, supra, guidance for in vitro skin absorption tests is available from OECD (2004) OECD Guideline for the Testing of Chemicals. Skin Absorption:in vitro method. Pages 1-8). Diffusion dells are commonly used to measure in vitro skin absorption, and these can be of the static type or flow-through type. The Franz diffusion cell has the following structures, in order from top to bottom: Donor compartment (containing test substance, such as a drug); Membrane (supporting membrane where skin is positioned); Receptor compartment with sampling port that allows access to receptor compartment; Surrounding lower half of receptor compartment is water jacket for maintaining temperature; At bottom of interior of receptor compartment, and in contact with fluid in receptor compartment, is magnetic stirrer. Optionally, the researcher can include dermal absorption tests with standards, such as benzoic acid, caffeine, and testosterone.

Also, according to Bartosova and Bajgar, supra, dose concentrations up to 10 mg/cm² or up to 10 microliters/cm² are used. The skin sample is equilibrated with receptor fluid for 10-30 minutes before applying dose to skin. Barrier integrity of skin is checked by methods that determine transepidermal water loss or transcutaneous electrical resistance. Kinetic parameters that can be determined include flux (J), permeability coefficient (Kₚ), and diffusion coefficient (D). When the testing period comes to its scheduled end, for example, after three hours or after 24 hours, some of the test substance may still located inside the skin, that is, in the membranes and cytosol of skin cells. Test substance inside the skin may optionally be included in the value for total substance that is absorbed.

Source of Human Skin

For CBD and THC, the inventor used for the receiving solution ethanol/water mix (30/70 by weight). The cadaver skin, the inventor received skin from a tissue bank such as Science Care in Phoenix Ariz. The donor human skin was dermatomed in Science Care to thickness of about 250 micrometers consisting of stratum corneum and part of epidermal layer and shipped to the inventor on dry ice.

After receiving the donor human skin, the inventor prepared the skin for testing in a Franz cell by thawing the skin to room temperature, washing in distilled water and cutting in round pieces to fit the diameter of the Franz cell opening. Before placing the patch on the human skin, the inventor dried the skin with a paper tissue. The skin with the attached patch was placed between the Upper Donor Chamber and the Lower Receiving Chamber and clamped tightly. After that, the receiving solution was filled into the Receiving Chamber making sure no bubbles are trapped beneath the skin. The test lasts typically 24 hrs and aliquots of 150 mL were withdrawn from the receiving chamber after different periods of time and analyze on HPLC. Display of the drug concentration in the receiving solution versus time was presented by graphs showing the kinetics of the transdermal passage of the drug from the patch through the skin into the receiving solution.

Measuring Thickness of Films and of Patches

Film thickness can be measured using puncture test and texture analyzer, such as Instron® 3366-2716015, Germany (see, Priya et al (2011) J. Pharm. Res. 3:56-65).

Patch thickness can be measured with a screw gauge, where thickness can be measured at various different spots on the patch. To measure surface pH, patch can be allowed to swell for 2 hours on the surface of an agar plate (2% w/v), and the pH then measured with pH paper. Swelling can be measured by taking the weight each hour for six hours, after placing patch on an agar plate (see, Velma et al (2014) Effect of novel mucoadhesive buccal patches of carvediol on isopenaline-induced tachycardia. J. Adv. Pharm. Technol. Res. 5:96-103). Residence time measured time that patch adheres to a mucosal membrane, where patch is glued to a substrate, with repeated up-and-down movement of the substrate until the patch detaches (see, Ismail et al (2003) Design and characteristics of mucoadhesive buccal patches containing cetyl pyridinium chloride. Acta Pharm. 53:199-212.

Dimple-Style Reservoir Vs. Balloon-Style Reservoir for Reservoir Patch Device

In a balloon embodiment, the present disclose can include a reservoir that is conformed like a sealed bag (or like a continual bag) or like a sealed balloon. In this embodiment, the reservoir is made of a material that is separate from backing and separate from permeable layer. In this embodiment, the reservoir may or may not be attached to backing or permeable layer by way of an adhesive or heat seal.

In a dimple embodiment, the reservoir has on a distal side a backing that has a dimple (or outpouching) where the dimple is conformed to hold drug, and where the reservoir has on proximal side a permeable layer. In other words, what prevents drug from spillout out of the outpouching is this permeable layer.

The backing and permeable layer are attached to each other, to prevent leaking of the drug. Attachment can be via an adhesive or heat-sealing. The present disclosure can exclude devices where this attachment is by adhesive, and can exclude devices where this attachment is by heat-sealing.

The present disclose can exclude devices with balloon reservoir. In other embodiments, the present disclosure can exclude devices with a dimple reservoir.

In the dimple embodiment, the permeable layer can comprise a plurality of slits, a plurality of tiny holes, or by being made of a porous layer. The present disclosure can exclude device with dimple reservoir.

Dimple reservoir device can include (or exclude) a layer that that resides in between drug and permeable layer. Also, dimple reservoir device can include (or exclude) a layer that resides on side of permeable layer facing the skin, where this layer is in substantial contact with the permeable layer. This layer that is on side of permeable layer of skin can be distal to adhesive layer and peelable backing layer.

Permeable layer can comprise permeable polypropylene film (US2006/0024520; US20016/115585), permeable polyethylene film (U.S. Pat. No. 4,793,003; WO2006/070672); permeable polyurethane film (U.S. Pat. No. 9,566,423).

Shapes of Reservoirs that Alter Delivery Rate Over the Course of Time

Reservoir of the present disclosure can be manufactured in predetermine shape, so that rate of release of an active agent to the skin or to a mucosal surface various over the course of hours, during the time frame when patch device is worn by a patient. For example, reservoir can be conical, where the wide surface (base of cone) is situated at the distal portion of patch device and where point of the cone is situated at the proximal portion of patch device. Proximal means the side of patch device closest to the skin, distal means the side of patch device farthest away from the skin. With cone reservoir, rate of drug transfer from patch to skin or mucosal surface gradually decreases over time. Reservoir can be hemispherical, resembling a gum drop, with base of gum drop closest to proximal side of patch device, and rounded surface of gum drop closest to distal side of patch device. Hemispherical reservoir gives initial rapid rate of drug release followed by rapid decrease in rate of drug relase. Reservoir can also have edges that are perpendicular to the skin-facing portion of the patch, that is, perpendicular to the peelable release of the dermal patch (in the event that the patch has a peelable release). See, U.S. Pat. No. 6,207,181 of Herrman, which is incorporated herein by reference in its entirety. The present disclosure can exclude a device with conical reservoir, with hemispherical reservoir, and/or hemispherical reservoir.

The reservoir device of the present disclosure can have only one conical reservoir, only two conical reservoirs, only three conical reservoir, at least one conical reservoir, at least two conical reservoirs, at least three conical reservoirs. The reservoir device can have only one, only two, only three, at least one, at least two, at least three hemispherical reservoirs. The reservoir device can have only one, only two, only three, at least one, at least two, at least three perpendicular sided reservoirs. Moreover, the reservoir device can have only conical reservoir(s), only hemispherical reservoir(s), only parallel sided reservoir(s), a combination of only conical reservoir(s) and hemispherical reservoir(s), a combination of only conical reservoir(s) and parallel side reservoir(s), a combination of only hemispherical and parallel side reservoir(s), or a combination of all three of conical, parallel side, and hemispherical reservoirs. The present disclosure also provides reservoirs of an ambiguous shape, such as that resembling a wrinkled, partially filled balloon, either alone or in combination with a conical, hemispherical, and/or parallel shaped reservoir. The present disclosure also provides reservoirs having the shape of a hot dog, either alone or in combination with a conical, hemispherical, and/or parallel shaped reservoir.

Fragile, Hollow Projections that Impair Tampering with Patch Device

In embodiments, the present disclosure provides, or can exclude, patch device that impairs attempts by user to physically extract active ingredient and/or that impairs attempts by users to extract by solvent active ingredient. Users may want to recover active ingredient from dermal patch for oral self-administration or for injection. Fragile, hollow projections preferrably occur as projections from main body of adhesive patch, for example, as projections from distal backing of adhesive patch (distal means side farthest from skin). The fragile hollow projections, which may resemble “cilia,” are manufactured so that they are more fragile than main body of adhesive patch or more fragile than any “walls” of adhesive patch. When user attempts to physically recover active ingredient, fragile projections are broken, releasing an antagonist of active ingredient. Also, when user attempts to use solvent to extract active ingredient, fragile projections dissolve (to some extent) releasing antagonist of active ingredient. Fragile, hollow projections are rigid and have thinner walls than walls of body of patch device, where goal is to ensure that physical tampering preferably ruptures the fragile, hollow projections (releasing antagonist) and promotes close vicinity and mixing of the released antagonist with the active ingredient. See, U.S. Pat. No. 7,740,879 of Royds and US2012/0238970 of Royds, which are incorporated herein by reference. In exclusionary embodiments, the present disclosure can exclude any patch device that comprises fragile, hollow projections, and/or can exclude projections dissolvable by solvent, and the like.

Rapidly Dissolving Films

Drug or other active substance can be provided in a rapidly dissolving film, for example, for a buccal patch or sublingual device serves a source of water, and where water is drawn into patch or device and dissolves film, resulting in release of drug from film and migration of drug to mucosal surface of user.

Films have been used as an alternative to pills and tablets. Film may contain an active ingredient, where active ingredient is uniformly distributed throughout film (US2005/0184427 of Yang, which is incorporated herein by reference in its entirety). Various techniques can be used to enhance uniformity, such as using a viscosity-increasing chemical as part of liquid composition of film, prior to drying the film. Another technique to enhance uniformity is drying the film in a way that avoids formation of a skin on the top surface of the film (where bottom surface rests on a surface or substrate). A problem with heat-drying, as with a forced air drier using hot air, is that the surface develops a skin. The skin blocks water from evaporating, resulting in water vapor building up inside, where the vapor rips open the film causing ripples and causing non-uniform drug distribution. Uniformity can be enhanced by pouring liquid film composition on a surface or substrate, and heating only the bottom (not the top) (see, U.S. Pat. No. 4,631,837 of Magoon). Alternatively or in addition to bottom heating, film matrix can be created by combining film-forming polymer with active ingredient and water, and also a polyhydric alcohol. Polyhydric alcohol increases viscosity. Polyhydric alcohols have the general formula HOCH₂(CHOH)nCH₂OH, and include sorbitol erythritol, glycerol, mannitol, and arabitol (Lin (1961) J. Biol. Chem. 236:31-36, Gerlsma (1968) J. Biol. Chem. 243:957-961). Film-forming polymers include, pullulan, hydroxypropylmethyl cellulose, hydroxethyl cellulose, polyvinyl pyrrolidone, carboxymethyl cellulose, and polyvinyl alcohol. In exclusionary embodiments, the present disclosure can exclude any device that comprises one or more of polyhedric alcohol, a film-forming polymer, or a film.

Emulsions and Self-Emulsifying Agents

The present disclosure provides emulsions, emulsifying agents, self-emulsifying agents, creams, and lotions. The following provides examples of self-emulsifying agents. Self-emulsifying drug delivery systems (SEDDS) and self-nano-emulsifying drug delivery systems (SNEDDS) have been reviewed (see, Cherniakov et al (2015) Expert Opin. Drug Deliv. 12:1121-1133). Self-emulsifying agents include glycerol monostearate, glycerol monooleate, and Cremophor RH40®. Cremophor RH40® is polyoxyl 40 hydrogenated castor oil. Cremophor EL® is polyoxyl 35 castor oil. These chemicals can be obtained from BASF Aktiengesellschaft, Ludwigshafen, Germany. In one aspect, the present disclosure can include formulations that comprise a self-emulsifying agent. In another aspect, the present disclosure can exclude formulations, and can exclude devices, that comprise a self-emulsifying agent.

Solubilizer SL-11 is a self-emulsifying agent that provides a nanoemulsion suitable for containing a hydrophobic drug (NOF America Corp., Irvine, Calif.). Emulsion with particle size under 50 nanometers can be made by these steps: (1) Dissolve drug in a suitable solvent, such as ethanol; (2) Add the drug solution prepared in (1) to Solubilizer SL-11, thoroughly mix to completely dissolve the contents; (3) The drug/SL-11 solution with solvent is made; (4) Evaporate the solvent at 50 degrees for about 1 hour to remove the solvent, or remove the solvent under a nitrogen stream; (5) Concentrated solution of SL-11 and the drug is made; (6) Soft capsules can be prepared by using the concentrated solution in (5) (NOF America Corp., Irvine, Calif.).

The following provides another non-limiting example. According to Shah et al (1994) Int. J. Pharmaceutics. 106:15-23, self-emulsifying agents can be made with polyglycolyzed glycerides (PGG) with varying fatty acid and polyethylene glycol (PEG) chain lengths, where these produce the self-emulsification of oil in water. The quality of the resulting emulsions depends on the oil and emulsifier pair selected and on the concentration of PGG as the emulsifier. One suitable oil is an oil with a medium-chain triglycerides (caprylic acid and capric acid; Neobee M5®). Another suitable oil is peanut oil. With formation of the emulsion, parameters that can be measured include droplet size distribution, droplet polarity, the release rate of the drug and the oil/water partition coefficient of the drug. PGG was found to be a workable emulsifiers for use in self-emulsifying drug delivery systems (SEDDS) (Shah et al (1994) Int. J. Pharmaceutics. 106:15-23).

Yet another non-limiting example of a self-emulsifying agent is provided by Chambin et al (2004) Int. J. Pharmaceutics. 278:79-89. This describes a self-emulsifying system using Gelucire® 44/14, an excipient from the lauroyl macrogolglycerides family. The laboratory method involves producing a fine oil-in-water emulsion when introduced into an aqueous phase under gentle agitation as SEDDS. The advantage is improved solubility and bioavailability of poorly water-soluble drugs. Gelucire® 44/14 was ground into a powder by cryogenic grinding to produce solid oral dosage forms and resulting in formulations made of Gelucire® 44/14 and ketoprofen (90/10). Cryogenic grinding produced Gelucire® 44/14 in a powder form, where this process did not change its physical properties, emulsification capacities and dissolution performances of the formulation tested.

Devani (2004) J. Pharmacy Pharmacology. 56:307-316, provide the following example, using the drugs danazol and mefenamic acid. In self-emulsifying drug delivery systems (SEDDS), drugs are dispersed in an oil-surfactant mix that emulsifies on contact with water. Self-emulsifying systems can be based on the Labrafil family of polyglycolysed oils, using Tween 80 and Tween 20 as surfactants. The more hydrophilic oil-surfactant mixes showed a greater ease of emulsification and a lower particle size. A linear relationship was observed between the hydrophile-lipophile balance (HLB) of the mix and the solubility of both danazol and mefenamic acid, with more hydrophilic mixes showing greater drug solubility values.

This provides another non-limiting example. Zupancic et al (2016) Eur. J. Pharm. Biopharm. 109:113-121 described emulsifying properties of SEDDS composed of long chain lipids (LC-SEDDS), medium chain lipids (MC-SEDDS), short chain lipids (SC-SEDDS) and no lipids (NL-SEDDS). The drug, enoxaparin was incorporated via hydrophobic ion pairing in the chosen SEDDS. The average droplet size of chosen LC-SEDDS, MC-SEDDS and NL-SEDDS ranged between 30 and 40 nm. MC-SEEDS containing 30% Captex 8000, 30% Capmul MCM, 30% Cremophor EL and 10% propylene glycol and NL-SEDDS containing 31.5% Labrafil 1944, 22.5% Capmul PG-8, 9% propylene glycol, 27% Cremophor EL and 10% DMSO exhibited 2-fold higher mucus diffusion than LC-SEDDS. Both MC-SEDDS and NL-SEDDS showed sustained in vitro enoxaparin release. Orally administrated MC-SEDDS and NL-SEDDS yielded an absolute enoxaparin bioavailability of 2.02% and 2.25%, respectively.

Further regarding emulsions, emulsifying agent can be characterized by Hydrophilic Lipophic Balance (HLB). HLB system is numbered 1 to 20. HLB values of 3 to 6 are lipophilic and these form water-in-oil emulsions (see, Vadlamudi, Hyndavi, and Tejeswari (2014) Current Drug Discovery Technologies. 11:169-180). HLB values of 8 to 18 are hydrophilic and these form oil-in-water emulsions (see, Grimberg, Nagel, and Aitken (1995) Environ. Sci. Technol. 29:1480-1487).

Permeation Enhancers

The present disclosure provides permeation enhancers, for example, for use with a dermal patch or for a buccal patch. Suitable permeation enhancers include, 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides (see, Shojaei et al (June 2001) Systemic drug delivery via the buccal mucosal route. Pharmaceutical Technology. Pages 70-81). Other enhancers of the present disclosure are 1-octanol, 2-ethylhexanol, 1-nonanol, 1-decanol, and so on.

Peroration enhancers of the present disclosure can be a biphasic composition having a lipid phase and a water phase. Lipid phase can be prepared by mixing isopropyl palmitate and lecithin. Water phase can be mixture of water and a surfactant. Surfactant can be Pluronic®, Pemulen®, Noveon®, or Carbopol®. Pemulen polymeric emulsifiers are high molecular weight, copolymers of acrylic acid and C10-C30 alkyl acrylate crosslinked with allyl pentaerythritol (Lubrizol, Inc. product sheet). Carbopol homopolymers are acrylic acid crosslinked with allyl sucrose or allyl pentaerythritol. Carbopol copolymer are acrylic acid and C10-C30 alkyl acrylate crosslinked with allyl pentaerythritol (Lubrizol, Inc. product sheet). Noveon® Polycarbophil, USP is a high molecular weight acrylic acid polymer crosslinked with divinyl glycol (Lubrizol, Inc. product sheet). Pluronic® polymers are block copolymers based on ethylene oxide and propylene oxide. They can function as antifoaming agents, wetting agents, dispersants, thickeners, and emulsifiers (BASF, Inc. product sheet). The present disclosure can exclude any formulation, composition, device, method, and such, that comprise one or more the molecules found in Pluronic®, Pemulen®, Noveon®, and Carbopol®.

PLOGel is “Pluronic Lecithin Organogel” (Pharmedica Enterprise, Selangor, Malaysia). PLOGel takes the form of an aqueous phase (240 mL poloxamer 407, potassium sorbate, water) and organic phase (60 mL lecithin, isopropyl palmitate, sorbic acid). The present disclosure can exclude any formulation, composition, device, method, and such that comprise one or more of PLOGel, poloxamer 407, potassium sorbate, isopropyl palmitate, sorbic acid, lecithin, and the like.

In exclusionary embodiments, the present disclosure can exclude any formulation, composition, device, method, and such that encompasses one of the above polymers, polymer compounds, and crosslinked polymer compositions.

In other exclusionary embodiments, the present disclosure can exclude compositions, formulations, dermal patches, layers, and the like, as well as methods, that comprise sulphoxides such as DMSO, Azones and Azone analogs such as laurocapram, transkarbams, 6-aminohexane acid esters, and can also exclude pyrrolidones such as 2-pyrrolidone, alcohols such as ethanol or decanol, glycols such as propylene glycol, surfactants, or vesicular carriers such as liposomes (see, Bartosova and Bajgar (2012) Curr. Med. Chem. 19:4671-4677).

Bioadhesive Materials

Bioadhesive polymer of the present disclosure, when swollen, creates a flexible network through with drug can diffuse. Bioadhesive material serves a matrix for retaining pharmaceutical agents, until patch is applied to the skin or to a mucosal surface of the consumer. Bioadhesive materials include, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene) (see, Shojaei et al (June 2001) Systemic drug delivery via the buccal mucosal route. Pharmaceutical Technology. Pages 70-81).

Dermal Patches with a Plurality of Adhesive Layers

The present disclosure provides dermal patches, laminated sheets, and related methods that comprise a plurality of adhesive layers. In one embodiment, a monolithic patch has these layers, from most distal to most proximal: (1) Backing; (2) Adhesive; (3) Carrier layer containing active agent; (4) Contact adhesive, and (5) Protective liner. In an exclusionary embodiment, the present disclosure can exclude this embodiment.

In another embodiment that is characterized by having a “rate controlling layer,” the monolithic patch has these layers, from distal to proximal: (1) Backing; (2) Adhesive; (3) Carrier layer containing active agent; (4) Adhesive layer; (5) Rate controlling polymer layer; (6) Adhesive layer; and (7) Protective liner. In an exclusionary embodiment, the present disclosure can exclude this embodiment.

The following concerns an embodiment where there is a “carrier layer” and where carrier layer is surrounded by and in contact with, on distal surface and on lateral surfaces, with an adhensive layer, and where carrier layer is surrounded by and in contact with, on proximal surface with “active ingredient permeable skin contact adhesive layer.” More generally, speaking present disclosure encompasses a “hat embodiment” taking the form of a dermal patch or other medical device where a first layer has a distal surface, proximal surface, and lateral surfaces. In this “hat embodiment” the distal surface, proximal surface, and lateral surfaces, are all surrounded by and in contact with a “hat layer.” The hat layer can be an adhesive layer or it can be an impermeable backing layer. The term “hat embodiment” and “hat layer” refer to the fact that the “hat layer” covers the first layer, in the same way that a man's hat covers the top of his head as well as his ears, forehead, and back of his head. The present disclosure provides a device with these layers, from distal to proximal: (1) Backing; (2) Adhesive; (3) Carrier layer; (4) Active ingredient permeable skin contact adhesive; and (5) Protective liner. In this embodiment, the “hat” can cover the lateral sides of the carrier layer and also cover the lateral sides of the “active ingredient permeable skin contact adhesive layer.” In an exclusionary embodiment, the present disclosure can exclude the above “hat” embodiment.

In another “hat” embodiment, the present disclosure provides, from distal to proximal: (1) Backing; (2) Adhesive layer; (3) Carrier layer; (4) Active ingredient permeable layer; (5) Rate-controlling polymer layer; and (6) Active ingredient permeable skin contact layer. The “hat” takes the form of backing plus adhesive layer, and they had covered the laterals sides of all four of these layers: carrier layer, active ingredient permeable layer, rate-controlling polymer layer, and active ingredient permeable skin contact layer. In an exclusionary embodiment, the present disclosure can exclude the above “hat” embodiment.

In other exclusionary embodiments, the present disclosure can exclude devices where: (1) Carrier layer is in direct and substantial contact with backing layer; (2) Carrier layer is in direct and substantial contact with an adhesive layer; (3) Adhesive layer is in direct and substantial contact with rate-controlling polymer layer; (4) An adhesive layer is in direct and substantial contact with protective liner; (5) Where the device comprises a “hat” configuration of layers; (6) Carrier layer is in direct and substantial contact with active ingredient permeable layer, (7) Active ingredient permeable skin contact layer is in direct and substantial contact with protective layer; (8) Active ingredient permeable skin contact layer is in direct and substantial contact with release liner or protective liner; (9) Where at least part of device has “hat” configuration and where only one layer is covered (surrounded on proximal face and on lateral faces) by the hat; (10) Where at least part of device has “hat” configuration and where only two layers are covered (surrounded on proximal face and on lateral faces) by the hat; (11) Where at least part of device has “hat” configuration and where only three layers are covered (surrounded on proximal face and on lateral faces) by the hat; (12) Where at least part of device has “hat” configuration and where only four layers are covered (surrounded on proximal face and on lateral faces) by the hat. The exclusionary embodiments of the present disclosure encompass any combination of the above exclusions. The above may apply to reservoir patches where, optionally, “reservoir” takes the place of “carrier layer.” Also, the above may apply to monolith patches.

Electrical Embodiments

The present disclosure encompasses skin patch devices with electric circuitry, for use in iontophoretic electroporative or electroporative delivery of active substances, such as drugs. The following arrangement uses electricity to drug out of “retainer” and into skin. The arrangement comprises the following, from distal-most layer to proximal-most layer. Distal means parts of patch farthest from skin and proximal means parts of patch closest to skin, when patch is placed adheringly to skin. The arrangement may be: (1) Conductive adhesive electrode that has a distal end (or layer) that sits on top of the stack of layers and proximal end that resides under most of the layers but sits on top of “retainer;” (2) Positive pole layer that may comprise manganese dioxide; (3) Electrolyte layer; (4) Negative pole layer that may comprise zinc powder; and (5) Medical electrode.” Medical electrode directly contacts skin, and current flows out of medical electrode through retainer (driving drug out of retainer and into skin), and then flows through skin, and to proximal end of conductive electrode. In the patch, no part of the conductive adhesive electrode touches the medical electrode. An insulator may be used to separate (in the structure of the patch) the conductive adhesive electrode from the medical electrode. Retainer can be a separate structure from patch, or retainer can be manufactured to be connected to patch. See U.S. Pat. No. 7,643,874 of Nitzan and US2010/0030129 of Nitzan, which are incorporated herein by reference in their entirety.

In exclusionary embodiments, the present disclosure can exclude any device that includes one or more electrodes. Also, disclosure can exclude any device where a reservoir (or matrix, layer, porous body, and such), that is manufactured separately from adhesive patch, and where user, e.g., physician or patient, assembles the reservoir with the patch, to create a stablely associated patch and reservoir.

Nutraceuticals and Pharmaceuticals

The present disclosure provides formulations, emulsions, and the like, as well as buccal patches and dermal patches, where the formulation, emulsion, buccal patch, and dermal patch, contains one or more of vitamin B1, vitamin D3, vitamin B12, or vitamin C, optionally in combination with one or more cannabinoids. Also, the formulation, emulsion, buccal patch, and dermal patch, can contain sildenafil.

Manufacturing Processes and Equipment

Sealing Two Strips Together at the Edges, and Coordinating Transverse Sealing to Create Pouches and Filling of the Pouches

What is provided is a method to feed two strips into a machine with rollers to move the strips at the same speed, and to cause the two strips to move downwards, where the first face of the first strip is caused to contact the first face of the second strip. The first face is caused to contact the first face of the second strip, in preparation for heating the edges of the two strips, thus sealing the two strips together, and in preparation for transverse heating, with heating at intervals of distance and time, thus creating a plurality of pockets in the sandwich of the two strips. When the two strips are moved downwards, the first strip and second strip are situated to form a thicker sandwich that moves downwards. Heaters resembling wheels or rollers, clamp down on the edges of the 2-strip sandwich, causing the 2-strip sandwich to form a long, closed tube. While the 2-strip sandwich moves downwards, what simultaneously occurs is simultaneous heating/sealing of a pair of transverse clamps. The transverse clamps create separate pouches in the long 2-strip sandwich. When the heated bars clamp down, what is created is a top seal of a previously-filled pouch, and the bottom seal of a pouch that has yet to be filled. Simultaneously occurring with heating/sealing at the edges by the heated wheels, and simulataneously occurring with heating by the transverse bars, is filling of each pouch as it is created, where filling is by a long tube that reaches down into the long sandwich to fill each pouch as it is created. See, U.S. Pat. No. 6,871,477 of Tucker, which is incorporated by reference in its entirety. The first strip can comprise an adhesive layer and permeable membrane, the second strip can be an impermeable backing, and the gel can comprise a cannabinoid in gel form.

Unrolling Three Different Layers from Rolls, Stripping Off Release Liners from Two of the Layers, Aligning the Three Layers Together to Form a Complex, and Rolling the Complex on to a Roll

The present disclosure provides machinery that can unroll a plurality of rolls, optionally with stripping off a release-layer from one or more of the rolls, and taking up the stripped-off release-layer on an empty rotating drum or roll. For example, three different rolls can contain three different laminates, the first laminate comprising: (1) Protective backing; (2) Combined zone of transport enhancement and zone of containment; and (3) Release layer. The second laminate can comprise: (1) Adhesive layer; (2) Zone of transport control; and (3) Release liner. And the third laminate can comprise: (1) Support film; (2) Adhesive; and (3) Removable liner that is not removed during the above-mentioned method. Machinery can include three rolls on three rotating mechanisms of first, second, and third laminate, respectively. Machinery can include take-up rolls for taking up release liners. Machinery can include a pair of rollers situated on opposite sides of moving sandwich of first laminate and second laminate for use in bringing the two laminates together. Also, machinery can include a pair of rollers situated on opposite side of the nacent 3-part sandwich, where the 3-part sandwich takes the form of the combined (in contact with each other) first and second laminate and the entering third laminate. The entering third laminate is simultaneously unrolled from its roll and then combined with the complex of first and second laminate. The final product is then moved, by way of pairs of rollers situated on opposite sides of the moving final product, and also moved by way of individual rollers, e.g., rollers called over roller, under roll, and over idler roller. The above-disclosed machinery can also include a device for sealing laminates together, a corona discharge for enhancing the sealing of the laminates together, a device for depositing a drug or adhesive or other composition on one or more of the laminates as the laminate is unrolled from its roll, and cutting devices for separating the sandwich of three laminates into patches. See, U.S. Pat. No. 5,370,924, which is incorporated herein by reference in its entirety.

Layered device may be assembled and then sealed by vacuum forming or by heat sealing without vacuum. In exclusionary embodiment, the present disclosure can exclude machinery, methods, and patches, made using one or more of vacuum forming, heat sealing, corona discharge, one or more crimp rolls, or cooperating nip.

Providing a Platen with Bar-Like Regions Separated by Channels, and Using the Platen to Stamp a Laminate, and to Provide Pressure on Regions that Need to be Collapsed, while Refraining from Providing Pressure on Regions that Contain Drug and Matrix

The present disclosure provides machinery, such as a platen with bar-like regions separated by channels, and where the bar-like regions are optionally heated. The platen can be used to selectively compress parts of a laminate, where the laminate (the “workpiece”) comprises an upper layer that is a cellular region and a lower layer that is a skin adhesive. The platen selectively compresses the distal sides (the right edge and left edge), resulting in collapse of the distal sides of the laminate. Optionally, only the part of the laminate destined to be collapsed is provided with the adhesive. The cellular region can be reticulated or it can be non-reticulated. The cellular region can be made of foamed thermoplastic resin. Cell size can be about 0.05, about 0.1, about 0.2, about 0.4, about 0.6, about 0.80, about 1.0, about 1.2, about 1.4, about 1.6, about 1.8, about 2.0, about 2.5, about 3.0, or about 4.0 millimeters. Collapsed regions are such that drug cannot easily pass through collapsed regions. In embodiments, non-collapsed central area (the area that resided under the channel during platen-manufacturing process) can contain a distally-situated layer of drug-releasing matrix (which contains drug) in contact with a proximally-situated layer of a medium through which drug can diffuse. The layer of medium through which drug can diffuse can be, e.g., gel, cream, or ointment. “Distal” means away from the skin when patch is attached to skin, and “proximal” means on the side of patch that is closest to skin, when patch is attached to skin. The compressed lateral parts of patch may be called “straps.” See, U.S. Pat. No. 5,505,958 of Bello, which is incorporated herein by reference in its entirety.

In exclusionary embodiments, the present disclosure can exclude patch devices with non-compressed cellular region, patch devices with compressed cellular region, layered structures with a distally-situated drug matrix and a proximally-situated gel, cream, ointment, or other medium through which drug can diffuse on its way to skin. Also, the present disclosure can exclude any composition, laminate, layered structure, and patch that was made via heating of a layered structure or via heating of a laminate.

Placing Drug Between Two Webs, Sealing Two Webs Together, Crimping the Sealed Webs to Form Pockets, and Cutting the Sealed Web

The present disclosure provides machinery and methods for using, as starting material, two different webs, each on a roller, where each web comprises one or more of a film, adhesive layers, impermeable layers, porous layers, and the like. The finished product takes the form of the two webs that are sealed together, and where an active ingredient, such as a composition comprising one or both cannabinoid and terpene, is contained therein. In the method, a first supply roll provides one web and a second supply roll provides second web. Machinery at various “stations” modify one of the webs or modify both of the webs, as the webs move along a conveyor belt. One station, which is optional, is a corona discharge station. The corona discharge modifies the surface chemistry of one or both of the webs, prior to marriage of the two webs together by operation of two crimp rolls. The corona discharge modifies the surface chemistry to improve adhesive properties of the first web and/or of the second web. Corona discharge is preferred where dissimilar materials (one material of first web, and other material of second web) are to be adhesively joined. Dissimilar materials can be, e.g., polyester polymer and ethylene acrylic acid polymer.

Another station is deposit station, which deposits active substance on one of the webs, as the web moves towards the crimp roll. Deposit station can include a reservoir that contains drug and tube leading from reservoir to location on web surface where drug is to be deposited. The deposit station preferrably occurs after the corona station. Also, the deposit station and corona station preferrably act on the same web, though optionally deposit station can operate on first web and corona station can operate on second web. The two webs are securely fastened together in a station taking the form of a first crimp roll and a second crimp roll. These rolls resemble gears, in that first crimp roll has projections and second crimp roll has depressions, which act meashingly in the manner of a “tongue-and-groove” to compress the two webs together and, at the same time, to stamp the joined webs into a pocket-like shape. The regions of the first crimp roll and second crimp roll that mesh together are called a “cooperating nip.”

Finally, after the webs pass through the corona station, drug deposit station, and crimp rolls, the joined webs are cut by rotary die cutter, to create flexible packages or flexible patches suitable marketing. Motors can drive rollers. Also, motors can drive drimp rolls. See, U.S. Pat. No. 4,782,647 of Williams which is incorporated herein by reference.

Separating Cut Patches from a Strip of Non-Cut Patches, and Transferring Cut Patches to a Carrier

This describes only one step in procedure for making adhesive patches, e.g., monolithic devices and reservoir devices. The procedure involves a cutter, transfer devices resembling wedges, and rollers. The rollers function to move a first web and a second web, in the manner of a conveyer belt. The first web takes the form of an auxiliary layer film on top, and then just under it, a drug-containing adhesive layer that sits on top of a carrier film. The first web, which has these three layers, is then later on supplemented by a process layer, where the result is a web consisting of four layers (process layer on top, then auxiliary layer film, then drug-containing adhesive layer, and on the bottom, carrier film). An earlier-occurring cutting process has cut the auxiliary layer film and the drug-containing adhesive layer into blocks. The first web is moved in one direction, e.g., to the left, and then with the help of the transfer devices resembling wedges, the squares are separated from the carrier film (the carrier film is then moved away to the right) and also separated from the combination of auxiliary layer film and process layer (which is moved upwards), where the squares end up residing on a carrier film. At this point the blocks are separated from each other, and any scrap that had been created with the cutting process is then discarded. This refers to the situation where cutting creates discrete blocks and creates scraps in between the blocks. The supporting film supports the blocks and moves away to the left. See, U.S. Pat. No. 6,059,913 of Asmussen, which is incorporated herein in its entirety.

Cutting Laminate to Create Fully Cut-Out Region and, within it, a Partially Cut (Scored) Region

Machinery, methods, and workpiece of the present disclosure comprises sheet of laminate, where shapes of the sheet (rectangles, ovals, circles) are cut fully through the laminate, and where the edges of the cut-out laminate is called, “periphery” (outer cut). Where the cut-out laminate is circular, the periphery is the same as the circumferential region. In addition to being cut at the periphery, the sheet is simulataneously cut during the cutting operation in a region within the periphery (inner cut). The inner cut has a smaller diameter than the outer cut. Also, the inner cut is to a shorter depth than the outer cut. In the case of a 3-layer laminate (release layer; pressure-sensitive adhesive, backing), the outer cut slices through all three layers, but the inner cut slices only partially through the top layer (the release layer). This partial cutting is more properly called, “scoring” rather than “cutting.” The goal of this 2-distance cutting method is to score the release layer to facilitate easy removal of the liner by the user, and at the same time, to avoid leaking of adhesive from the patch during storage of the patch. Machinery for the 2-distance cutting method can take the form of a roller covered with cutting stampers (similar to cookie-cutters). Each cookie cutter stamps all the way through the laminate. Within each cookie cutter resides a second (smaller diameter) cookie cutter which is sized so that it only cuts partially through the top layer of the laminate (thus only scoring the top layer). In an alternative machinery, a first roller bears an array of only the larger diameter (and longer cutting distance) cookie cutters, while the second roller bears an array of the smaller diameter (and scoring distance) cookie cutters. In operation, the two rollers operate simultaneously, and the cookie cutters on the first roller are aligned exactly with the cookie cutters on the second roller and, in operation the cutting (cutting through all layers) occurs simultaneously with scoring, for each patch. See, U.S. Pat. No. 5,656,285 of Sablotsky, which is incorporated by reference in its entirety. In addition to the one cutting roller (or to the two cutting rollers), the machinery can have a pressure roller and a support roller, for use in driving the sheet of laminate. In exclusionary embodiments, the present disclosure can exclude an adhesive dermal patch that has a scored region, such as a scored release layer.

Efficient Separation of Punched Patches from Scrap Web

During manufacture of adhesive patches, patches are stamped out from, or cut from, a sheet consisting of various layers. These layers may include backing, matrix containing a drug, skin adhesive, and release layer. During cutting, some of the punched patches that have not yet been separated from scrap web may cling to the scrap web as the scrap web is pulled away from the sheet. Where this clinging is maintained as the scrap web is pulled away, the result will be undesirable discarding of the clinging punched patches along with the scrap web. This type of undesired clinging can be increased by flow of adhesive out of the edges of the punched patch, followed by flow of the adhesive to contact the scrap web. Efficient separation of punched patches can be accomplished by way of a probe or probes that contact the punched patch and shove the punched patch on to a horizontally moving conveyor belt as the scrap web is drawn upwards for eventual discard. The probe can take the form of a rotating roller where the roller is covered with blocks having the same shape and exactly the same dimensions (or dimensioned to be about 5% smaller, about 10% smaller, about 15% smaller, about 20 smaller, and the like, in area, as compared to the punched patch). The blocks can have a shape, as viewed from “above,” that is square, rectangular, oval, round, etc., and to have a shape corresponding to the punched patch. Thus, as the roller rotates, each block presses down on a corresponding punched patch (as the punched patch continues to move on the conveyor belt) while the scrap web is simultaneously detached and drawn upwards by the rotation of the roller. An alternative to using a roller covered with block probes, is a roller covered with flexible bristles. As the roller rotates, the bristles press springfully down on the punched patches, the bristles remaining bent, causing the punched patches to separate from the scrap web. At the same time, the bristles pressing on the scrap web are greatly bent at first, but as the scrap web is pulled upwards, the bristles spring out to their full (un-bent) length. See, US2017/0136648 of Grader, which is incorporated herein by reference in its entirety. In an exclusionary embodiment, the present disclosure can exclude manufacturing machinery and methods, comprising a roller with blocks or a roller with bristles, for use in preventing punched patches from adhering to the scrap web.

### Making Powders and Gels

The present disclosure provides a method for making powders, comprising combining and mexing a cannabinoid such as THC with one or more of ethanol, PEG, isopropylmyristate, carbopol, triethanol, permeation enhancer, acetone, and water, and then drying using heat, then milling to form a powder, and then optionally adding a lower alcohol such as ethanol, propanol, or butanol, to form a gel.

### Preventing Formation of Drug Crystals

If undesired crystals form in any solution, such as solution for adhesive, solution for adhesive/drug mixture, or solution for drug matrix for placing in a reservoir, heating steps are available to prevent such crystals. The procedure for casting a film can involve use of a casting solution, a drying oven, a laminator, and an annealing oven. “Annealing” refers to heating followed by cooling to room temperature. What can get annealed is a liquid dispersion or an article formed from liquid dispersion. Undesired crystals can occur after die-cutting has occurred. Undesired crystals can occur even after removing water with dessicants. A method that avoids formation of drug crystals can involve casting the reservoir solution to form a drug reservoir film. Drug reservoir film can be on a backing of aluminized polyethylene terephthalate (Scotchpak®). After casting, the composition on the backing is put in an oven to remove solvent, e.g., chloroform, where the temperature is greater than melting point of the drug's crystals. This is followed by packaging, and further heating (see, e.g., U.S. Pat. No. 7,169,409 of Dohner, which is incorporated by reference in its entirety). First heating can be at, e.g., about 50, about 55, about 60, about 65, about 70, about 75, about 80, about 85, about 90, about 95 degrees C. Second heating can be at, e.g., about 50, about 55, about 60, about 65, about 70, about 75, about 80, about 85, about 90, about 95 degrees C. First heating step and second heating step can each be selected from one of 10 min, 15 min, 20 min, 30 min 40 min, 60 min, 4 hours, 8 hours, 12 hours, 16 hours, 20 hours, or 24 hours.

Exclusionary Embodiments

The present disclosure can exclude a composition, formulation, dermal patch, methods of use, methods of manufacture, that comprise one or more of the following: capsaicin, 2-arachidonylglycerol, curcumin, glycerylmonooleate, glycerylmonostearate, lecithin, acacia gum, xylitol, carboxymethylcellulose, a self-emulsifying agents, glycerol monostearate, glycerol monooleate, Cremophor RH40®, Cremophor EL®, hydroxypropyl cellulose, carbopol, poly(vinyl pyrrolidone), sodium carboxymethyl cellulose, hydroxyethyl cellulose, polycarbophil, pectin, chitosan, xanthan gum, locust bean gum, hydroxypropyl methylcellulose, poly(vinyl alcohol), poly(isoprene), poly(isobutylene). The present disclosure can also exclude one or more of, 23-lauryl ether, Aprotinin, Azone, Benzalkonium chloride, Cetylpyridinium chloride, Cetyltrimethylammonium bromide, Cyclodextrin, Dextran sulfate, Laurie acid, Laurie acid/propylene glycol, Lysophosphatidylcholine, Menthol, Methoxysalicylate, Methyl oleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides, and Alkyl glycosides. What can also be excluded is a formulation, composition, device, or method, that comprises pre-gelatinized starch, gelatinized starch, gelatinized corn starch, glycogelatin, alpha-tocopherol, glycogelatin, hemp oil, THC, CBD, gum acacia, sorbitol, xylitol, soy lecithin, a complex of two different gels (one with net negative charge and the other with net positive charge), and a compositions that comprise a solvent with a cosolvent.

What can be excluded is pharmaceutical compositions with 1-5% enhancer. What can be excluded is pharmaceutical compositions with 0.5-5% neutralizer, or with any amount of neutralizer. What can be excluded is compositions with greater than 0%-5% by weight isopropyl myristate, or with any amount thereof. What can be excluded is pharmaceutical compositions with 0%-10% by weight carbopol, or with any amount of carbopol. What can be excluded is pharmaceutical compositions with about 10% ethanol, about 15%, about 20%, about 24%, about 30%, about 35%, about 40%, about 45%, about 50%, about 55%, about 60%, about 65%, about 70%, about 75%, about 80%, about 85%, about 90% ethanol.

For delivery of cannabinoids, for example, a system of solvent/cosolvent can be ethanol (solvent)/propylene glycol (cosolvent). Solvents can be anhydrous alcohol, ethanol, propanol, or isopropanol. Cosolvent can be propylene glycol or PEG. Ratio of solvent/cosolvent (by weight) can be about 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like. In exclusionary embodiments, the present disclosure can exclude solvent/cosolvent compositions where the ratio is, 5/95, about 10/90, about 15/85, about 20/80, about 25/75, about 30/70, about 35/65, about 40/60, about 45/55, about 50/50, about 55/45, about 60/40, about 65/35, about 70/30, about 75/25, about 80/20, about 85/15, about 90/10, about 95/5, and the like.

Excluding Structures

The present disclosure can exclude a adhesive patch device, buccal patch device, sublingual drug delivery device, that that has more than one reservoir. The patch device of the present disclosure can have only one reservoir, only two reservoirs, only three reservoirs, only four reservoirs. The present disclosure can exclude microneedles, and can exclude a patch device that has microneedles. The present disclosure can exclude any adhesive patch device, buccal patch device, sublingual drug delivery device, that comprises a bilaminate layer, that comprises a trilaminate layer, that comprises a tetralaminate layer. Also, the present disclosure can exclude a bilaminate layer, exclude a trilaminate layer, and exclude a tetralaminate layer.

Further Exclusionary Embodiments

What can be excluded is a formulation with an ethanol content, by weight, of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%. Also, what can be excluded is a formulation with an ethanol content, by weight, that encompasses about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%. Moreover, what can be excluded is a formulation with an ethanol content, by weight, that encompasses (range that equals or range that includes) a range that is 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%. What can also be excluded is a device that encompasses one or more of the above formulations.

The present disclosure can provide a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5. In exclusionary embodiments, what can also be excluded is a formulation that comprises ethanol and propylene glycol (or glycerol monostearate, or glycerol monooleate, or monoglyceride, or diglyceride, or triglyceride, or PEG, or phospholipid, or surfactant), and where the ratio (weight/weight basis) is about 5/95, 10/90, 15/85, 20/80, 25/75, 30/70, 35/65, 40/60, 45/55, 50/50, 55/45, 60/40, 65/35, 70/30, 75/25, 80/20, 85/15, 90/10, or 95/5.

Formulations with specific concentrations, on a weight basis, of propylene glycol or of any other compound can be excluded. What can be excluded are formulations containing about 0.1%, of about 0.2%, of about 0.4%, of about 0.6%, of about 0.8%, of about 1.0%, of about 2%, of about 4%, of about 6%, of about 8%, of about 10%, of about 15%, of about 20%, of about 25%, of about 30%, of about 35%, of about 40%, of about 45%, of about 50%, and the like, of propylene glycol, polyethylene glycol (PEG), polyalkylene glycol, ethanol, emulsion (e.g., oil droplets in water, water droplets in oil, liposome suspension), colloid, solvent, penetration enhancer, stabilizing agent, solubilizing agent (e.g., surfactant, detergent), gelling agent (either in dry state or in hydrated state), hydrogel (either in dry state or in hydrated state), adhesive, or any other compound, can be excluded.

Also, what can be excluded are formulations that encompass (range that equals or range that includes) the range of 0-0.1%, 0-5%, 0-10%, 0-20%, 0-30%, 0-40%, 0-50%, 5-10%, 5-15%, 5-20%, 5-40%, 5-50%, 10-20%, 10-30%, 10-40%, 10-50%, 10-60%, 10-70%, 20-30%, 20-40%, 20-50%, 20-60%, 20-70%, 20-80%, 30-40%, 30-50%, 30-60%, 30-70%, 30-80%, 40-50%, 40-60%, 40-70%, 40-80%, 40-90%, 50-60%, 50-70%, 50-80%, 50-90%, 60-70%, 60-80%, 60-90%, 60-100%, 70-80%, 70-85%, 70-90%, 70-95%, 70-100%, 80-85%, 80-90%, 80-95%, 80-100%, 85-90%, 85-95%, 85-100%, and the like, of propylene glycol, polyethylene glycol (PEG), ethanol, emulsion (e.g., oil droplets in water, water droplets in oil, liposome suspension), colloid, solvent, penetration enhancer, stabilizing agent, solubilizing agent (e.g., surfactant, detergent), gelling agent (either in dry state or in hydrated state), hydrogel (either in dry state or in hydrated state), adhesive, or any other compound. In another aspect, the present disclosure can include (encompass, comprise) a formulation, composition, device, or method that comprises one or more of the above chemicals, at any of the recited “about” values, and at any of the recited ranges.

Without implying any limitation, the present disclosure can exclude a composition that comprises one or more of the following compounds, and can also exclude a device that comprises one or more of the following compounds. What can be excluded is a compound that is, buprenorphine, clonidine, estradiol, fentanyl, granisetron, methylphenidate, nitroglycerin, oxybutynin, scopolamine, selegiline, testosterone, a vaccine, influenza virus vaccine, a mammalian hormone, a synthetic analogue of a mammalian hormone, a chemically modified mammalian hormone, lidocaine, estrogen, salicyclic acid, a contraceptive, rivastigmine, rotogotine, tulobuterol, adrenergic agonist, cholinesterase inhibitor, dopamine receptor agonist, oxybutynin, bupropion, varenicline, nicotine, antidepressant, smoking cessation drug, cholinsterase inhibitor, methylphenidate, buprenorphine, opioid analgesic agent, sumatriptan, antiviral drug, anti-retrovirus drug, mammalian steroid, chemical analogue of mammalian steroid, drug for attention-deficit hyperactivity disorder, and so on.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) CBD. Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups.

What can be excluded is an adhesive polymer, or a device comprising an adhesive polymer, where the adhesive polymer has over 1 free hydroxyl groups per 100 atoms of the adhesive polymer.

What can be excluded is an adhesive polymer, or a device comprising an adhesive polymer, where the adhesive polymer has over 5 free hydroxyl groups per 100 atoms of the adhesive polymer.

Also, what can be excluded is an adhesive polymer, or a device comprising an adhesive polymer, where the adhesive polymer has over 10 free hydroxyl groups per 100 atoms of the adhesive polymer

Moreover, what can be excluded is an adhesive polymer, or a device comprising an adhesive polymer, where the adhesive polymer has over 20 free hydroxyl groups per 100 atoms of the adhesive polymer, and so on.

In embodiments, what can be excluded is a monolith-type device where a backing is not in direct contact with a matrix of skin adhesive; where matrix of skin adhesive is not in direct contact with a releasable liner; where matrix does not comprise CBD; or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

What can also be excluded is a preparation, or a device comprising a preparation, where the preparation has over 1% penetration enhancer, over 2%, over 3%, over 4%, over 5%, over 6%, over 7%, over 8%, over 9%, over 10%, over 12%, over 14%, or over 16%, of penetration enhancer. Also, what can be excluded is a preparation, or a device comprising a preparation, where the preparation has under 1% penetration enhancer, under 2%, under 3%, under 4%, under 5%, under 6%, under 7%, under 8%, under 9%, under 10%, under 12%, under 14%, or under 16%, of penetration enhancer.

In other embodiments, what can be excluded is a preparation, a composition, a device comprising a preparation, a device comprising a composition, where said preparation or composition has a CBD (or THC, or combined weight of CBD and THC) content by weight of under 1%, under 2%, under 3%, under 4%, under 5%, under 6%, under 8%, under 10%, under 12%, under 14%, under 16%, under 18%, under 20%, under 25%, under 30%, under 35%, under 40%, under 45%, under 50%, under 55%, under 60%, under 65%, under 70%, under 75%, and so on. Also, what can be excluded is a preparation, a composition, a device comprising a preparation, a device comprising a composition, where said preparation or composition has a CBD (or THC, or combined weight of CBD and THC) content by weight that is greater than 5%, greater than 6%, greater than 7%, greater than 8%, greater than 10%, greater than 12%, greater than 14%, greater than 16%, greater than 18%, greater than 20%, greater than 25%, greater than 30%, greater than 35%, greater than 40%, greater than 45%, greater than 50%, greater than 55%, greater than 60%, greater than 65%, greater than 70%, and so on. In embodiments, what can be excluded is a preparation, a composition, a device comprising a preparation, or a device comprising a composition, where the percent by weight is defined by one or more of the above “under” or “greater than” parameters. “Composition” can refer to, for example, matrix of a skin adhesive, or to fluid in hydrophilic porous membrane, and so on. Alternatively, the present disclosure can comprise one or more of the above compositions, as set forth by “under” parameters or “greater than” parameters.

Moreover, in embodiments what can be excluded is any device that does not include an occlusive system polymer film, that does not include a polyethylene occlusive polymer film, that does not include a PET occlusive polymer film, that does not include an occlusive polymer film made of both polyethylene and PET. Also, what can be excluded is a device that has an overlay patch, and a device that does not comprise an overlay patch.

In embodiments, polar organic liquid can comprise, or can exclude, one or more of methanol, ethanol, propanol, isopropanol, butanol, pentanol, acetic acid, propionic acid, butyric acid, valeric acid, caproic acid, caprylic acid, capric acid, lauric acid, myristic acid palmitic acid, stearic acid, palmitoleic acid, oleic acid, linoleic acid, linolenic acid, linear alkanes of 5, 6, 7, 8, 9, 10, 11, 12, 13, 14, 15, 16, 17, 18, 19, 20, 21, 22, 23, 24, or more carbons, branched chain alkanes with a backbone of 5, 6, 7, 8, 9, 10, 11, 12, 13, 14, 15, 16, 17, 18, 19, 20, 21, 22, 23, 24, or more carbons, linear alkenes (olefins) of 5, 6, 7, 8, 9, 10, 11, 12, 13, 14, 15, 16, 17, 18, 19, 20, 21, 22, 23, 24, or more carbons, branched chain alkenes (olefins) with a backbone of 5, 6, 7, 8, 9, 10, 11, 12, 13, 14, 15, 16, 17, 18, 19, 20, 21, 22, 23, 24, or more carbons, and so on. Alternatively, the present disclosure can comprise one or more of the above polar organic liquids.

The present disclosure can exclude a composition, device, method, that comprises an essential oil, a plant oil, a vegetable oil, or a fish oil. Also, the present disclosure can exclude a composition, device, method, that comprises one or more terpenes. What can be excluded is a composition, device, method, that comprises one or more of peppermint oil, orange oil, lemon oil, cannabis oil, hemp oil, and so on. Also, what can be excluded is any composition, device, or method, that comprises one or more of alpha-bisabolol, borneol, alpha-caryophyllene, beta-caryophyllene, elemene (alpha, beta, gamma, or delta), limonene, camphene, camphor, delta-3-carene, caryophyllene oxide, alpha-cedreen, citral, eucalyptol, beta-eudesmol, eudesm-7(11)-en-4-01, farnesene, fenchol, alpha-guaiene, geraniol, guaiol, germacrene B, guaia-1(10)-11-diene, humulene, alpha-humulene, isoborneol, linalool, menthol, myrcene, alpha-myrcene, beta-myrcene, nerol, cis-ocimene, trans-ocimene, alpha-phellandrene, alpha-pinene, beta-pinene, pulegone, sabinene, alpha-terpinene, alpha-terpineol, terpinolene, terpineol, thymol, trans-2-pinanol, selina-3,7(11)-diene, or valencene.

Also, what can be excluded is a formulation, composition, device, lozenges, or sublingual pill that comprises one or more of sodium phosphate, potassium phosphate, guar gum, gum arabic, locust bean gum, xanthan gum, carrageenan, carob gum, ghatti gum, pectin, tragacanth gum, acacia gum, mannitol, sorbitol, lactose, modified lactose, maltitol, mannitol, magnesium stearate, hydroxypropylmethylcellulose film, non-crystallizing sugar, or non-crystallizing sugar alcohol.

What can be excluded is any formulation, composition, device, method, and such, that comprises menthol and isopropyl myristated in one of the following ratios (weight/weight): 200/10, 180/10, 160/10, 140/10, 120/10, 100/10, 90/10, 80/10, 70/10, 60/10, 50/10, 40/10, 30/10, 20/10, 15/10, 10/10, and so on, or one of the following ratios: 10/10, 10/15, 10/20, 10/30, 10/40, 10/50, 10/60, 10/70, 10/80, 10/90, 10/100, 10/120, 10/140, 10/160, 10/180, 10/200, and so on. Also, what can be excluded are compositions defined by a range of any of the above two ratio values. Also, what can be excluded is any formulation, composition, device, method, and such, that comprises menthol and isopropyl myristated in one of the following ratios (weight/weight): about 200/10, about 180/10, about 160/10, about 140/10, about 120/10, about 100/10, about 90/10, about 80/10, about 70/10, about 60/10, about 50/10, about 40/10, 30/10, 20/10, 15/10, 10/10, and so on, or one of the following ratios: about 10/10, about 10/15, about 10/20, about 10/30, about 10/40, about 10/50, about 10/60, about 10/70, about 10/80, about 10/90, about 10/100, about 10/120, about 10/140, about 10/160, about 10/180, about 10/200, and so on. Also, what can be excluded are compositions defined by a range of any of the above two ratio values.

In device embodiments, a device of the present disclosure is substantially free of all cannabinoids that are not CBN. In composition embodiments, a composition of the present disclosure is substantially free of all cannabinoids that are not CBN.

In device embodiments, a device of the present disclosure is substantially free of one or more of tetrahydrocannabinol (THC), tetrahydrocannabinolic acid (THC-a), cannabinol (CBN), and cannabichromene (CBC). Also, a device of the present disclosure is substantially free each and every one of tetrahydrocannabinol (THC), tetrahydrocannabinolic acid (THC-a), cannabinol (CBN), and cannabichromene (CBC). In composition embodiments, a composition of the present disclosure is substantially free of one or more of tetrahydrocannabinol (THC), tetrahydrocaimabinolic acid (THC-a), cannabinol (CBN), and cannabichromene (CBC). Also, a composition of the present disclosure is substantially free each and every one of tetrahydrocannabinol (THC), tetrahydrocannabinolic acid (THC-a), cannabinol (CBN), and cannabichromene (CBC).

In one aspect, the term “substantially free” can mean that the quantity of one or more of THC, THC-a, CBN, and CBC, occurs at a molar quantity that is under 20%, under 15%, under 10%, under 5%, under 4%, under 2%, under 1%, under 0.5%, under 0.1%, under 0.05%, or under 0.01%, that of CBD. In another aspect, the term “substantially free” can mean that the quantity of each and every one of THC, THC-a, CBN, and CBC, occurs at a molar quantity that is under 20%, under 15%, under 10%, under 5%, under 4%, under 2%, under 1%, under 0.5%, under 0.1%, under 0.05%, or under 0.01%, that of CBD.

The following methods of measurement take into account the physical nature of a composition and the physical nature of the container or matrix that comprises a composition. In measuring a composition that is “substantially free,” what can be measured is all compounds that are comprised by the composition, where the composition takes the form of an oil, a paste, a slurry, an adhesive, a powder, a solution, and the like, or that takes the form of a matrix, a reservoir, and impregnated fabric, a flask, a conduit, that holds, contains, absorbs, adsorbs, and the like, the oil, a paste, a slurry, an adhesive, a powder, a solution, and the like.

Without implying any limitation, the present disclosure can exclude a composition that comprises one or more of the following compounds, and can also exclude a device that comprises one or more of the following compounds. What can be excluded is a compound that is, buprenorphine, clonidine, estradiol, fentanyl, granisetron, methylphenidate, nitroglycerin, oxybutynin, scopolamine, selegiline, testosterone, a vaccine, influenza virus vaccine, a mammalian hormone, a synthetic analogue of a mammalian hormone, a chemically modified mammalian hormone, lidocaine, estrogen, salicyclic acid, a contraceptive, rivastigmine, rotogotine, tulobuterol, adrenergic agonist, cholinesterase inhibitor, dopamine receptor agonist, oxybutynin, bupropion, varenicline, nicotine, antidepressant, smoking cessation drug, cholinsterase inhibitor, methylphenidate, buprenorphine, opioid analgesic agent, sumatriptan, antiviral drug, anti-retrovirus drug, mammalian steroid, chemical analogue of mammalian steroid, drug for attention-deficit hyperactivity disorder, and so on.

In embodiments, the present disclosure can exclude a reservoir-type device where backing does not directly contact reservoir; or where reservoir does not directly contact a hydrophilic porous membrane; or where hydrophilic porous membrane does not directly contact a release liner; or where reservoir does not contain all of: (1) a liquid carrier, (2) a gelling agent, and (3) CBD. Also, what can be excluded is a reservoir-type device that does not comprise all of the above.

In embodiments, what can be excluded is an adhesive polymer, or a device comprising an adhesive polymer, where the adhesive polymer reacts with amines. Also what can be excluded, is an adhesive polymer, or a device comprising an adhesive polymer, where the adhesive polymer has any free hydroxyl groups, where the adhesive polymer has over 1 free hydroxyl groups per 100 atoms of the adhesive polymer, where the adhesive polymer has over 5 free hydroxyl groups per 100 atoms of the adhesive polymer, where the adhesive polymer has over 10 free hydroxyl groups per 100 atoms of the adhesive polymer, where the adhesive polymer has over 20 free hydroxyl groups per 100 atoms of the adhesive polymer, and so on. For this exclusionary embodiment, the skilled artisan understands that any polymer consists of a large number of atoms, for example, about five thousand atoms.

In embodiments, what can be excluded is a monolith-type device where a backing is not in direct contact with a matrix of skin adhesive; where matrix of skin adhesive is not in direct contact with a releasable liner; where matrix does not comprise CBD; or all of the above.

What can also be excluded is a preparation, or a device comprising a preparation, where the preparation has over 1% gelling agent, over 2%, over 3%, over 4%, over 5%, over 6%, over 7%, over 8%, over 9%, over 10%, over 12%, over 14%, or over 16%, of gelling agent. Also, what can be excluded is a preparation, or a device comprising a preparation, where the preparation has under 1% gelling agent, under 2%, under 3%, under 4%, under 5%, under 6%, under 7%, under 8%, under 9%, under 10%, under 12%, under 14%, or under 16%, of gelling agent.

What can also be excluded is a preparation, or a device comprising a preparation, where the preparation has over 1% penetration enhancer, over 2%, over 3%, over 4%, over 5%, over 6%, over 7%, over 8%, over 9%, over 10%, over 12%, over 14%, or over 16%, of penetration enhancer. Also, what can be excluded is a preparation, or a device comprising a preparation, where the preparation has under 1% penetration enhancer, under 2%, under 3%, under 4%, under 5%, under 6%, under 7%, under 8%, under 9%, under 10%, under 12%, under 14%, or under 16%, of penetration enhancer.

In other embodiments, what can be excluded is a preparation, a composition, a device comprising a preparation, a device comprising a composition, where said preparation or composition has a CBD (or THC, or combined weight of CBD and THC) content by weight of under 1%, under 2%, under 3%, under 4%, under 5%, under 6%, under 8%, under 10%, under 12%, under 14%, under 16%, under 18%, under 20%, under 25%, under 30%, under 35%, under 40%, under 45%, under 50%, under 55%, under 60%, under 65%, under 70%, under 75%, and so on. Also, what can be excluded is a preparation, a composition, a device comprising a preparation, a device comprising a composition, where said preparation or composition has a CBD (or THC, or combined weight of CBD and THC) content by weight that is greater than 5%, greater than 6%, greater than 7%, greater than 8%, greater than 10%, greater than 12%, greater than 14%, greater than 16%, greater than 18%, greater than 20%, greater than 25%, greater than 30%, greater than 35%, greater than 40%. greater than 45%, greater than 50%, greater than 55%, greater than 60%, greater than 65%, greater than 70%, and so on. In embodiments, what can be excluded is a preparation, a composition, a device comprising a preparation, or a device comprising a composition, where the percent by weight is defined by one or more of the above “under” or “greater than” parameters. “Composition” can refer to, for example, matrix of a skin adhesive, or to fluid in hydrophilic porous membrane, and so on. Alternatively, the present disclosure can comprise one or more of the above compositions, as set forth by “under” parameters or “greater than” parameters.

Moreover, in embodiments what can be excluded is any device that does not include an occlusive system polymer film, that does not include a polyethylene occlusive polymer film, that does not include a PET occlusive polymer film that does not include an occlusive polymer film made of both polyethylene and PET. Also, what can be excluded is a device that has an overlay patch, and a device that does not comprise an overlay patch.

In embodiments, polar organic liquid can comprise, or can exclude, one or more of methanol, ethanol, propanol, isopropanol, butanol, pentanol, acetic acid, propionic acid, butyric acid, valeric acid, caproic acid, caprylic acid, capric acid, lauric acid, myristic acid palmitic acid, stearic acid, palmitoleic acid, oleic acid, linoleic acid, linolenic acid, linear alkanes of 5, 6, 7, 8, 9, 10, 11, 12, 13, 14, 15, 16, 17, 18, 19, 20, 21, 22, 23, 24, or more carbons, branched chain alkanes with a backbone of 5, 6, 7, 8, 9, 10, 11, 12, 13, 14, 15, 16, 17, 18, 19, 20, 21, 22, 23, 24, or more carbons, linear alkenes (olefins) of 5, 6, 7, 8, 9, 10, 11, 12, 13, 14, 15, 16, 17, 18, 19, 20, 21, 22, 23, 24, or more carbons, branched chain alkenes (olefins) with a backbone of 5, 6, 7, 8, 9, 10, 11, 12, 13, 14, 15, 16, 17, 18, 19, 20, 21, 22, 23, 24, or more carbons, and so on. Alternatively, the present disclosure can comprise one or more of the above polar organic liquids.

### Inhaling Embodiments

Aerosols and dry powder formulations for inhaling are available. See, Mitchell, Nagel, Wiersema, and Doyle (2003) AAPS PharmSciTech. 4(4) Article 54 (9 pages); Asai et al (2016) Pharm. Res. 33:487-497; Kopsch et al (2017) Int. J. Pharm. 529:589-596; Fishier and Sznitman (2017) Inhalation. 11:21-25. Vaporizers are available, for example, from Storz and Bickel (Tuttlingen, Germany), Arizer Tech (Waterloo, Canada), Organicex (Las Vegas, Nev.), and Elemental Technologies (Seattle, Wash.).

### Examples

Monolithic Patch Embodiments, as Described in US2017/0071870 of Weimann

The following writing is from US2017/0071870 (Ser. No. 15/265,823) of Weimann, which is incorporated herein by reference in its entirety.

Transdermal monolithic CBD patch formulation: Adhesive polymer: 60-95, CBD: 5-20 Penetration enhancer: 0-20, Adhesive polymer: Acrylate from Hankel, Silicone from Dow Corning. PIB from BASF CBD: pure crystalline powder Penetration enhancer: Oleic acid, isopropyl palmitate (IPP), dimethylsulfoxide (DMSO), 1,2-propylene glycol (1,2-PG), isopropylmyristate (IPM). In this example, the dry adhesive matrix is 30-50 micrometers thick. The area of the patch can be square or oval. The best size of the patch is 20 cm² by 40 cm².

In a monolithic design, a release liner is coated with a mixture of CBD and a PIB or amine-compatible silicone skin adhesive laminated to the backing material. How the Monolithic CBD Patch Works: Step 1. CBD is dissolved in ethyl alcohol or 1,2 PG and mixed into the adhesive solution and penetration enhancer is added if needed. Step 2. Adhesive mix is dispensed on the release liner by means of “knife-over-roll” coating method and dried in the oven at drying time from 1 min to 3 min or until all residual solvents are below 1 ppm. Step 3. Dried adhesive film is laminated to the backing film by means of nipping and edges are slit for farther die cutting of the patches. Step 4. The laminate is placed on the die cutting machine and proper size patches are cut and later packaged in the pouches and boxes.

How CBD is Delivered from Patch Formulation to the Body Through the Skin

Formulations of monolithic patches were prepared by solubilizing CBD in different adhesives and CBD transdermal flux was performed through human cadaver skin using Franz Diffusion Cell method. We found the highest transdermal flux of CBD from a formulation that comprises PIB adhesive and 10% CBD. This shows that a patch measuring 20 cm² can deliver a daily systemic dose of about 5 mg of CBD.

Exemplary Monolithic Patch Invention Formulations: Formulation 1. 10% CBD in EtOH. Formulation 2. 10% CBD in EtOH/H₂O (9/1). Formulation 3. Penetration enhancers: 1,2 PG, IPP, oleic acid, DMSO.

In a first monolithic-style device, a skin adhesive is mixed with the CBD to define a monolithic mixture of adhesive and CBD. The skin adhesive is coated on a backing that is preferably occlusive. The skin adhesive is preferably an amine-compatible silicone adhesive.

In a second monolithic-style device, a skin adhesive is mixed with the CBD (which may be present as substantially pure CBD or an oil extract of a cannabis plant which comprises CBD and other cannabinoids) to define a substantially monolithic mixture of adhesive and CBD. The skin adhesive is preferably a polyisobutylene adhesive having a viscosity-average molecular weight ranging from about 30,000 Daltons to about 70,000 Daltons, preferably, from about 35,000 Daltons to about 65,000 Daltons, and more preferably from about 40,000 Daltons to about 60,000 Daltons.

Manufacturing Method for Monolithic Patch

CBD is dissolved in ethyl alcohol or 1,2-propylene glycol (1,2 PG) and mixed into the adhesive solution and penetration enhancer is added if needed. Adhesive mix is dispensed on the release liner by means of “knife-over-roll” coating method and dried in the oven at drying time from 1 min to 3 min or until all residual solvents are below 1 ppm. Dried adhesive film is laminated to the backing film by means of nipping and edges are slit for further die cutting of the patches. The laminate is placed on the die cutting machine and proper size patches are cut and later packaged in the pouches and boxes.

Referring to FIG. 2 of U.S. Pat. No. 10,272,125 of Weimann, an example of monolithic-style transdermal drug delivery device 40 for delivering CBD is depicted. Monolithic transdermal device 40 includes a backing 42 of the type described previously with respect to backing 22 of reservoir transdermal device 20. A matrix 44 of skin adhesive mixed with a therapeutically effective amount of CBD is coated on one side of backing 42. The matrix 44 is preferably formulated to adhere the device 20 to the user's skin for a period of no less than about 24 hours while avoiding appreciable skin irritation to the user's skin. A release liner 48 is releasable adhered to matrix 44 on a surface of matrix 44 opposite the surface adhered to backing 42. First side 49 of release liner 48 faces away from matrix 44 and a portion of second side 51 of release liner 48 is adhered to matrix 44. To use the monolithic transdermal device 40, the release liner 48 is peeled away and the exposed surface of adhesive matrix 44 is applied to the skin.

The skin adhesive comprising matrix 44 preferably comprises at least one of an acrylate pressure sensitive adhesive, a polyisobutylene pressure sensitive adhesive, and an amine-compatible silicone pressure sensitive adhesive. Suitable acrylate adhesives include DuroTak 87-2516. Suitable polyisobutylene adhesives include those having a viscosity-average molecular weight ranging from about 30,000 Daltons to about 70,000 Daltons, preferably from about 35,000 Daltons to about 65,000 Daltons, and more preferably from about 40,000 Daltons to about 60,000 Daltons.

Matrix 44 preferably comprises a polyisobutylene adhesive having a viscosity-average molecular weight as described above and an adhesion/viscosity modifier. The adhesion/viscosity modifier is preferably a mineral oil or silicone fluid present in an amount ranging from about one (1) to about ten (10) percent by weight of matrix 44, more preferably from about two (2) to about six (6) percent by weight of matrix 44, and still more preferably from about three (3) to about four (4) percent by weight of the matrix 44. Mineral oils that are suitable for use as the adhesion/viscosity modifier have a molecular weight ranging from 100 to about 1000 Daltons, more preferably from about 200 to about 600 Daltons, even more preferably from about 350 Daltons to about 450 Daltons, and still more preferably about 400 Daltons. Silicone fluids that are suitable for use as the adhesion/viscosity modifier preferably comprise —OH end-capped polydimethylsiloxanes having a kinematic viscosity at 20.degree. C. ranging from about 100 cSt to about 1000 cSt. Commercially available silicone fluids that may be used as the adhesion/viscosity modifier include the Dow Corning Q7-9120 fluids, which are available in kinematic viscosities (at 20.degree. C.) of 20, 100, 350, 1000, and 12,500 cSt. In preferred examples of silicone adhesion/viscosity modifier, the Q7-9120 100 cSt or 1000 cSt (or mixtures thereof) are used.

Preferred polyisobutylene adhesives are not supplied with mineral oil. In certain preferred examples, the polyisobutylene component of matrix 44 is a Vistanex LM polyisobutylene adhesive. In other preferred examples, the polyisobutylene component of matrix 44 is an Oppanol B13 polyisobutylene adhesive supplied by BASF.

In another example, the adhesive component of matrix 44 may comprise a blend of acrylic adhesive and polyisobutylene adhesive, and preferably, a blend of an acrylic adhesive and a polyisobutylene adhesive having the viscosity-average molecular weight described above (from about 30,000 Daltons to about 70,000 Daltons, preferably from about 35,000 Daltons to about 65,000 Daltons, and more preferably from about 40,000 Daltons to about 60,000 Daltons). When acrylic adhesives are combined with such polyisobutylene adhesives, the amount of acrylic adhesive by weight of the total amount of adhesive in matrix 44 is preferably from about one percent to about 50 percent. In one example, the adhesive component of matrix 44 comprises 80 percent Oppanol B 13 by weight of the total amount of adhesive in matrix 44 and twenty percent Durotak 87-2516 by weight of the total amount of adhesive in matrix 44.

Monolithic device may also include one or more penetration enhancers, including oleic acid, isopropyl palmitate (IPP), DMSO, 1,2 propylene glycol, and isopropyl myristate (IPM). The amount of penetration enhancer preferably ranges from zero to about ten (10) percent by weight of the matrix. In an exclusionary embodiment, the present disclosure can exclude any patch or any formulation that has more than one type of penetration enhancer

The skin contact area of device is preferably at least about 10 cm², more preferably at least about 15 cm², and still more preferably at least about 18 cm². At the same time, the skin contact area of device is preferably no more than about 30 cm², preferably no more than about 25 cm², and still more preferably no more than about 22 cm². At a given flux rate, the skin contact area may be selected to achieve the desired daily dose of CBD (or the dose over whatever time period is of therapeutic interest). The above writing is from U.S. Pat. No. 10,272,125 of Weimann.

Knife-Over-Roll Coating

Without implying any limitation on the present invention, knife coating is a process by which a thin liquid coating is formed on a continuous web by the application of an excess of coating liquid which is subsequently metered by a rigid knife held in close proximity to a rigidly supported web. The thickness of the coating depends primarily on the clearance, or gap, between the knife and the web, and upon the geometry of the gap (bevel angle, length). Roll coating is a process by which a thin liquid film is formed on a continuous web by use of two or more rotating rolls, such that the fluid flow in a small gap between a pair of rotating rolls is the primary factor controlling the thickness and uniformity of the coated film. The thickness of the coating depends primarily on the gap between adjacent rolls and their relative speeds. Two basic types of roll coaters are distinguished by the relative direction of roll surface motion in the gap: in forward roll coating the roll surfaces move in the same direction and in reverse roll coating they move in opposite directions. In terms of the flow fields, knife coating is a subset of forward roll coating where one surface is stationary. See, Coyle, D. J (1997) Knife and Roll Coating in Liquid Film Coating (ed. S. F. Kistler and P. M. Schweizer). Chapman & Hall, London; W. Rehnby, M. Gustafsson, M. Skrifvars (June 2008) Conference Paper, Coating of Textile Fabrics with Conductive Polymers for Smart Textile Applications, pages 100-103.

### Example of Reservoir Patch Embodiments

Reservoir Patch Manufacturing Method

The present inventor has used the following manufacturing method for reservoir patch, where the method used the indicated stages:

STAGE 1. Gel dispenser. Dispensing active gel solution on membrane. At this point, the stage of manufacture can be represented by an isolated blob of active gel.

STAGE 2. Heat seal press. Gel is covered with heat sealable film and heat seal around the gel. At this point, the stage of manufacture can be represented by a blob in the center, surrounded by a layer of film.

STAGE 3. Kiss cut press. Kiss cutting along the periphery of the heat seal ring. At this point, the stage of manufacture can be represented by a blob at the center, surrounded by a layer of film and where the interior side of the layer is intact and where the exterior layer is perforated. According to one source, “Kiss cutting is . . . a method for providing a converted adhesive tape solution. During the kiss-cutting process, the perimeter of each piece is stamped out by a sharp metal die or by a precision laser beam . . . the cut does not penetrate the piece's backing material (liner). Even though the die or laser makes a clean cut all the way through the usable portion of the material, it merely “kisses” the liner sheet. This allows single or multiple adhesive materials to remain on a liner sheet or roll until the end user is ready to remove them.” (CAN-DO® National Tape, Nashville, Tenn.). According to another source, “Laser kiss cutting is used to cut the top layer of a material without cutting through an attached material. Sticker labels are a good example of laser kiss cutting in action. In this process, the outline of the label can be cut without cutting the release or backing material. Typically, CO₂ lasers are used for kiss cutting applications. Laser kiss cutting can also be combined with perforating or “through cutting” on a single application. (Preco Kansas, Lenexa, Kans.).

STAGE 4. Kiss cut press. Reservoir is covered with overlay film, and kiss cut along the overlay periphery. At this point in the manufacture, the article of manufacture obtained in STAGE 3 is surrounded by another layer, where this layer is the overlay film.

STAGE 5. Cut through press. Patch is cut through the overextended release liner, for easier peel. At this point, the article of manufacture resembles that obtained in STAGE 4, except that the article of manufacture is chopped into segments, where each segment is suitable for attaching to the skin of a human patient or a human consumer.

Reservoir Patch as Described in US2017/0071870 of Weimann

The following writing, and structure numbers, are from U.S. Pat. No. 10,272,125 of Weimann, which is incorporated herein by reference in its entirety. Referring to FIG. 1 of U.S. Pat. No. 10,272,125, a reservoir-style transdermal delivery device 20 for the transdermal delivery of CBD is depicted. Reservoir-style transdermal delivery device 20 comprises a backing 22 and a hydrophilic, porous membrane 24. The backing 22 and hydrophilic, porous membrane 24 are attached to one another so as to define a closed volume which acts as a reservoir 26. A preparation 27 comprising CBD, a liquid carrier, and a gelling agent is disposed in the reservoir 26. First side 34 of the hydrophilic, porous membrane 24 is in contact with the preparation 27. A second side 36 of the hydrophilic, porous membrane 24 faces away from backing 22 and is coated with a skin adhesive 30. The skin adhesive 30 is preferably formulated to adhere the device 20 to the user's skin for a period of no less than about 24 hours while avoiding appreciable skin irritation to the user's skin. Preferred skin adhesives 30 include amine-compatible, silicone, pressure sensitive adhesives. In certain examples, an amine-compatible silicone skin adhesive 30 is provided which comprises a trimethylsiloxy end-capped reaction product of a silanol end-blocked polydimethylsiloxane and a silicate resin. The skin adhesive is preferably provided as an organic solvent solution comprising from about 50 percent to about 70 percent by weight of solid adhesive in an organic solvent like heptane or ethyl acetate and having a viscosity at 20 degrees C. of from about 400 mPa-s to about 1300 mPa-s, preferably from about 450 mPa-s to about 1250 mPa-s, and more preferably from about 500 mPa-s to about 1200 mPa-s.

A first surface 29 of a release liner 28 is releasably adhered to skin adhesive 30, and a second surface 31 of release liner 28 faces away from skin adhesive 30. Suitable release liners include occlusive polymeric films, such as polyester, polypropylene, coated with a release coating that is releasably adherable to silicone, polyisobutylene, and silicone adhesives. Suitable release coatings on first surface 29 of release liner 28 include fluoropolymers and silicone polymers. Commercially-available, coated release liners that are suitable for use as release liner 28 include Scotchpak 1022, 9741, 9744, 9748, and 9755 supplied by 3M of Minneapolis, Minn., and FRA 314 and 315 supplied by Fox River Co. To use the reservoir transdermal device 20, release liner 28 is peeled away from skin adhesive 30, thereby exposing skin adhesive 30, and the device 20 is applied so that the skin adhesive 30 contacts the user's skin.

Suitable examples of such amine-compatible silicone adhesives include the BIO-PSA 7-4301 and 7-4302 skin adhesives supplied by Dow Corning. BIO-PSA 7-4301 is a high tack, amine-compatible silicone adhesive in heptane available with a solids content of 60 percent and 70 percent and corresponding viscosities at 20.degree. C. of 450 mPa-s and 1600 mPa-s. BIO-PSA 7-4302 is a high tack, amine-compatible silicone adhesive in ethyl acetate with a solids content of 60 percent by weight and a viscosity of 1200 mPa-s at 20 degrees C. The skin adhesive 30 is coated to a thickness per unit area on the membrane 24 that is preferably from about 10 to about 20 g/m², more preferably from about 12-18 g/m², and still more preferably from about 14-16 g/m².

Hydrophilic, porous membrane 24 preferably has a mean flow pore size of no more than about 1 micron, preferably not more than about 0.8 microns, still more preferably no more than about 0.4 microns, and even more preferably no more than about 0.2 microns. At the same time, porous membrane 24 preferably has a mean flow pore size of no less than about 0.02 microns, more preferably no less than about 0.04 microns, still more preferably no less than about 0.06 microns, and even more preferably no less than about 0.08 microns. The mean flow pore size may be determined in accordance with the method set forth at page 17, line 22 to page 18, line 4 of published PCT Application WO2010072233.

In the same or other examples, hydrophilic porous membrane 24 preferably has a porosity of at least about 60 percent, more preferably at least about 65 percent, and still more preferably at least about 70 percent. At the same time, hydrophilic porous membrane 24 preferably has a porosity of no more than about 90 percent, more preferably no more than about 85 percent, and still more preferably no more than about 80 percent. Porosity values may be calculated as described at page 7, lines 24 to 27 of WO2010072233.

In the same or other examples, hydrophilic porous membrane 24 preferably has a thickness of no more than about 50 microns, preferably no more than about 40 microns, and even more preferably no more than about 35 microns. At the same time, hydrophilic porous membrane 24 preferably has a thickness of no less than about 10 microns, more preferably no less than about 20 microns, and still more preferably no less than about 25 microns. Membrane thicknesses may be determined as described at page 18, lines 19-21 of WO2010072233.

In the same or other examples, hydrophilic porous membrane 24 preferably has an air permeability as determined by the Gurley Test Method (according to ISO 5636-5) that is preferably at least about 10 sec/50 ml, more preferably at least about 20 sec/50 ml, and still more preferably at least about 25 sec/50 ml. At the same time, hydrophilic porous membrane 24 preferably has an air permeability of no more than about 50 sec/50 ml, more preferably no more than about 40 sec/50 ml, and still more preferably no more than about 35 sec/50 ml.

In the same or other examples, hydrophilic porous membrane 24 preferably has a tensile strength in the machine direction as determined by ASTM D882-12 that is preferably at least about 10 MPa, more preferably at least about 15 MPa, and still more preferably at least about 20 MPa. In the same or other examples, the hydrophilic porous membrane 24 preferably has a percent elongation in the machine direction as determined by ASTM D882-12 that is preferably at least about 10 percent, more preferably at least about 15 percent, and still more preferably at least about 20 percent.

Hydrophilic porous membrane 24 preferably comprises at least one polymeric material. In one example, hydrophilic porous membrane 24 comprises a polyolefin polymer and a hydrophilic component that comprises a hydrophilic polymer and optionally, a surfactant. As used herein, the term “hydrophilic” when used to describe a porous membrane refers to a membrane that at 20.degree. C. provides a water flux for demineralized water through the membrane of at least 0.5 liters/(m²hbar).

The content of the polyolefin polymer is preferably less than or equal to 98 percent by weight based on the total dry weight of the membrane 24, and the content of the hydrophilic component(s) is preferably at least 2 weight percent based on the total dry weight of the membrane. In certain preferred examples, the membrane is formed by combining the polyolefin polymer with the hydrophilic components(s) and optional additives with a solvent to form a blend in the form of a gel, a solution, or a homogeneous mixture, followed by extruding the blend. Suitable polyolefins (such as polyethylene), hydrophilic components, and additives are described in WO2010072233.

In another preferred embodiment, device comprises transdermal patch formulation comprising a reservoir in the shape of a “ravioli” constructed with microporous hydrophilic or hydrophobic membrane on one side and occlusive film on other side.

In embodiments, device comprises transdermal reservoir patch formulation as thixotropic alcohol or alcohol/water solution gelled with hydroxyalkyl cellulose containing CBD at high concentration ranging from 1% to 50% CBD Moreover, device comprises transdermal reservoir patch formulation comprising a reservoir containing thixotropic alcohol or alcohol/water solution gelled with hydroxyalkyl cellulose and containing CBD at a high concentration, ranging from 1% to 50% and skin penetration enhancers in a concentration range of 0% to 10%.

What is also encompassed, is transdermal patch formulation comprising a reservoir in shape of “ravioli” constructed with microporous hydrophilic or hydrophobic membrane on one side and occlusive film on other side where the microporous membrane is coated with thin layer of silicone adhesive. In delivery embodiments, reservoir patch of 20 cm.sup.2 is capable of systemically delivering CBD at about 0.5 mg/day, about 1.0 mg/day, about 1.5 mg/day, about 2.0 mg/day, about 5.0 mg/day, about 10 mg/day, about 15 mg/day, about 20 mg/day, about 25 mg/day, about 30 mg/day, about 35 mg/day, about 40 mg/day, and the like.

In other delivery embodiments, reservoir patch of 20 cm.sup.2 is capable of systemically delivering CBD at least 0.5 mg/day, at least 1.0 mg/day, at least 1.5 mg/day, at least 2.0 mg/day, at least 5.0 mg/day, at least 10 mg/day, at least 15 mg/day, at least 20 mg/day, about 25 mg/day, about 30 mg/day, at least 35 mg/day, at least 40 mg/day, and so on.

### Example of Pill Formulations (Sildenafil, Cannabidiol)

Sublingual pill formulation was developed and tested for the active ingredients, cannabidiol and sildenafil. The formulation of the pill was: disintegrating agent (9 grams); microcrystalline cellulose (24 grams); saccharin sodium (0.75 grams); Mannitol (100 grams); magnesium stearate (1.5 grams). Active ingredients: 15 grams (CBD, Sildenafil). Total (150.5 grams).

Pill formulation was developed to meet the acceptable performance criteria such as: Hardness, Friability and Disintegration; Hardness (greater than 4 kg/cm²); Friability (less than 2%); Disintegration (less than 100 sec). Sublingual pills were made using a manual pill press.

The laboratory results were as follows. Sildenafil Citrate Sublingual Pill Performance: Diameter (0.6 cm); Thickness (0.435 cm); Average Weight (124.5 mg); Hardness (10.3 kilograms/cm²); Friability (0.6%) Disintegration (90 sec). Cannabidiol Sublingual Pill Performance: Diameter (0.6 cm); Thickness (0.435 cm); Average Weight (134.1 mg); Hardness (8 kilograms/cm²); Friability (0.2%); Disintegration (75 sec). Cannabidiol was sourced from hemp extract in crystalline form having purity 99.8% (0.00% THC).

In the inventor's experience with the sublingual pills formulation is that we are formulating for one concentration of the pill active ingredient. The main idea is to find the best set of excipients that will provide fast disintegration with a acceptable hardness. The goal is as follows: pill of 130 mg containing 10 mg of active ingredient; or pill formulation of 240 mg containing 20 mg of active ingredient. Keeping the active ingredient constant the inventor can test time of disintegration as a function of the concentration of magnesium stearate. Regarding cannabidiol (CBD), that amount of cannabidiol (CBD) will be 10 mg and the total mass of the pill will be always 130 mg. The inventor possesses a machine that is capable to make only that size of pill. Regarding sildenafil, the same goes for sildenafil: 10 mg sildenafil and 130 mg total mass of the pill with varied amount of magnesium stearate. The variable will be magnesium stearate and another ingredient that will offset the changes in weight of magnesium stearate.

Suppliers: Disintegrating agent (Pharmaburst 500 from SPI Pharma, Wilmington, Del.); microcrystalline cellulose (Avicel 102 from FMC BioPolymer); saccharin sodium (Spectrum Chemical MFG. Corp.); mannitol (from RPI Research Products International); magnesium Stearate (Spectrum Chemical MFG. Corp.). Sildenafil and cannabinoids are available from, for example, Sigma-Aldrich, St. Louis, Mo. For testing pills and tablets, friability, hardness, dissolution, and disintegration can be assessed by equipment from Copley Scientific, Ltd., Nottingham, UK. Equipment includes Friabiity Tester Series FR (FR1000, FR2000, Friabimat SA400), disintegration tester (DTG1000, DTG2000, DTG4000), and dissolution apparatus (basket, paddle, paddle over disk, cylinder, and vertical diffusion cell (Franz cell)). Friability is the tendency for a tablet to chip, crumble, or break under compression. Pharmaburst 500 contains mannitol, sorbitol, crospovidone, silica, aspartame, and magnesium stearate (see, H. Kathpalia and K. Jogi. Co-processed excipients. A review. World J. Pharma. Res. 3:3863-3885).

This provides laboratory data on CBD sublingual pill and sildenafil sublingual pill. In testing each of these types of pills, one variable is amount of disintegration agent, and another variable is amount of microcrystalline cellulose. Constant parameters are saccharin sodium, mannitol, magnesium stearate, and active ingredient. Experiment involves one set of formulation batches that include CBD, and another set of formulation batches that include seldenafil. For each of these two types of batches, the quantities are as follows: Disintegrating agent (0, 3, 6, 9, 12, 15, and 18 grams disintegrating agent); microcrystalline cellulose (33, 30, 27, 24, 21, 18, and 15 grams microcrystalline cellulose), saccharin sodium (0.75 grams), mannitol (100 grams), magnesium stearate (1.5 grams), active ingredient (CDB or sildenafil) (15 grams). The total weight of a batch is 150.5 grams.

### Examples of Adhesives, Tackifiers, and Enhances, with Reference to Data in US2017/0071870 of Weimann

Adding acrylic adhesive with non-functionality Durotak 87-900A and adhesive with only OH-functionality Durotak 87-2510 compared with PIB with enhancers azone and oleic acid and DMSO. Azone is “1-dodecyl azepan-2-one.”

PIB adhesive with tackifiers that improve adhesion to skin using acrylic pressure sensitive adhesive mixed in at 1-50%. Also use of cycloaliphatic hydrocarbon resins such as Escorez 5300® resins from ExxonMobil. The disclosure provides a graph showing peel strength from skin.

PIB adhesive with enhancers: at 3% of azone or oleic acid double the transdermal delivery from PIB. The disclosure provides a graph showing transdermal flux.

### Examples of Hemp Oil, Hydrogels, CBD, and THC, with Reference to Data in US2017/0071870 of Weimann

Use of hemp oil with CBD of high concentration 80-95% containing different terpenes improves transdermal delivery of CBD. The disclosure provides a graph of transdermal flux from matrix with crystalline CBD vs. matrix with hemp oil of 80% CBD.

Delivery of CBD and THC from semisolid Hydrogels saturated with CBD and THC oils of high concentration of CBD and THC 80-95%. Oils are saturated in mix with EtOH/water in ratio 80/20 also with enhancers azone, oleic acid and limonene. The disclosure provides a graph of transdermal flux.

Show THC oils of high concentration of THC (80-95%) mixed with 1-20% EtOH or with EtOH/water 80/20 (1-10%) in reservoir patch delivering high transdermal doses of THC. Addition of more than 10% of ethanol lowers the flux. The disclosure provides a graph of transdermal flux.

### Examples from US2017/0071870 of Weimann

CBD Patch with Menthol, Camphor and Salicylic acid.

### Example

CBD patch with 0.01% Capsicum.

### Example

CBD with nutraceutically active ingredients.

### Example

CBD/THC ratio 1/1 in patch produces 2/1 transdermal dose ratio. The disclosure provides a graph of transdermal flux.

### Drug and Nutraceutical Examples

The disclosure provides Melatonin Patch, Lidocaine Patch, Menthol, Camphor, Salicylic Acid Patch, Hang Over patch with Dihydromyricetin, Vitamin B1 patch, Vitamin D3 patch, Vitamin B12 patch, Vitamin C patch, Sildenafil sublingual pill, Sildenafil fast dissolving strip, Sildenafil buccal patch, Cannabinol sublingual pill, Cannabinol fast dissolving strip, Cannabinol buccal patch, and the like.

Dermal Patch Flux Data of the Present Disclosure

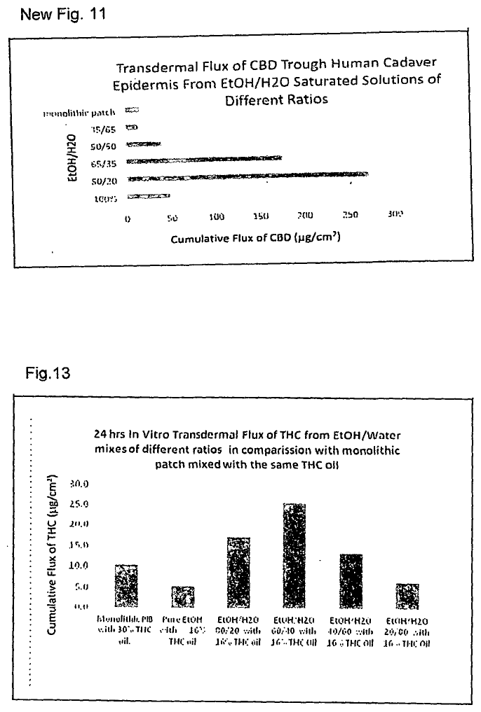

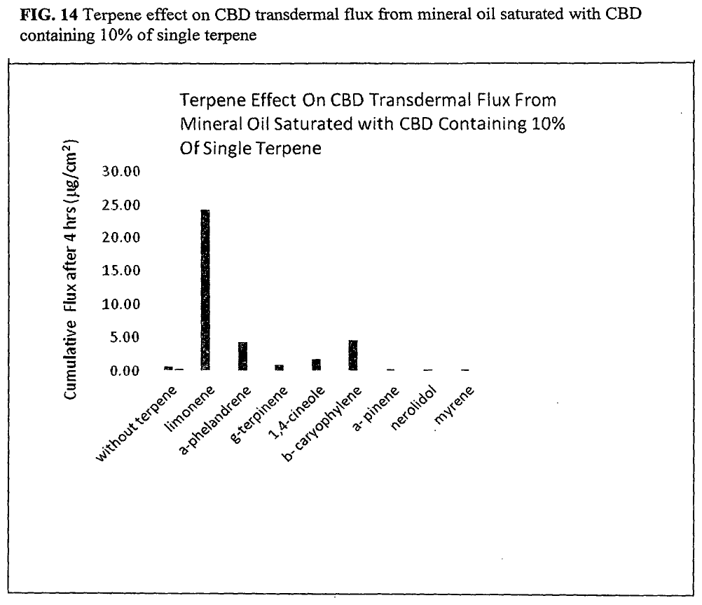

FIG. 13 shows comparison of 24 hours In Vitro flux of THC from different hydrogel matrices in comparison with monolithic patch mixed with THC oil. FIG. 13 shows of 24 hours in vitro transdermal flux of THC from EtOH/Water mixes of different ratios in comparison with monolithic patch mixed with the same THC oil. The results of FIG. 13 indicate that the THC flux from reservoir patch depends on the ethanol/water ratio. The greatest THC flux was observed when the ratio of ethanol to water was 60/40 saturated with THC and the THC flux was about 2.5 time greater than from monolithic patch. For FIG. 13, the data bar on the left is from a monolithic patch with PIB. Liquid hydrogel containing 60% EtOH and 40% water mixed with THC oil delivers 3 times greater flux of THC than monolithic patch with same concentration of THC.

The three data bars on the right are from reservoir patches. These reservoir patches did not contain any adhesive. However, when applied to the skin these reservoir patches are held in place on the skin by an overlay patches.

In embodiments, reservoir patch uses EtOH/water ratio of about 5/95 (vol/vol), about 10/90 (vol/vol), about 15/85 (vol/vol), about 20/80 (vol/vol), about 25/75 (vol/vol), about 30/70 (vol/vol), about 35/65 (vol/vol), about 40/60 (vol/vol), about 45/55 (vol/vol), about 50/50 (vol/vol), about 55/45 (vol/vol), about 60/40 (vol/vol), about 65/35 (vol/vol), about 70/30 (vol/vol), about 75/25 (vol/vol), about 80/20 (vol/vol), about 85/15 (vol/vol), about 90/10 (vol/vol), about 95/5 (vol/vol), and the like. In exclusionary embodiments, the present disclosure can exclude any reservoir patch that contains EtOH/water at any of these ratios, and can exclude any reservoir formulation with EtOH/water at any of these ratios. In embodiments, these particular ratios can be used to define ratios of any other solvents, such as the ratio of dimethylsulfoxide/water.

Also, in embodiments, reservoir patch uses EtOH/water ratio where ethanol percentage (vol/vol) is between 0.1-5% EtOH with the rest water, between 5-10% EtOH with the rest water, between 10-15% EtOH with the rest water, between 15-20% EtOH with the rest water, between 20-25% EtOH with the rest water, between 25-30% EtOH with the rest water, between 30-35% EtOH with the rest water, between 35-40% EtOH with the rest water, between 40-45% EtOH with the rest water, between 45-50% EtOH with the rest water, between 50-55% EtOH with the rest water, between 55-60% EtOH with the rest water, between 60-65% EtOH with the rest water, between 65-70% EtOH with the rest water, between 70-75% EtOH with the rest water, between 75-80% EtOH with the rest water, between 80-85% EtOH with the rest water, between 85-90% EtOH with the rest water, between 90-95% EtOH with the rest water, between 95-99.5% EtOH with the rest water, and so on. In exclusionary embodiments, the present disclosure can exclude any reservoir that contains EtOH/water at any of these ratio ranges (or at any combination or sum of these ratios), and can exclude any reservoir formulation with EtOH/water at any of these ratio ranges. In embodiments, these particular ratio ranges can be used to define ratio ranges of any other solvents, such as the ratio of dimethylsulfoxide/water.

Dermal Patch Data of the Present Disclosure

FIG. 11 shows Transdermal Flux of CBD Trough Human Cadaver Epidermis from EtOH/Water Saturated Solutions for Different Ratios. FIG. 11 shows results of in vitro transdermal flux of CBD from different ethanol/water solutions of different ratios as used in reservoir patch construction. The flux of CBD from solution of ethanol water at ratio 80/20 and saturation concentration of CBD was, dramatically and unexpectedly, about 10 times greater than from a monolithic patch. The present disclosure therefore discloses that, reservoir patch formulations prepared from ethanol/water mixes saturated with cannabinoids such as CBD and THC and other ones, deliver greater transdermal fluxes than monolithic patches.

Dermal Patch Data of the Present Disclosure

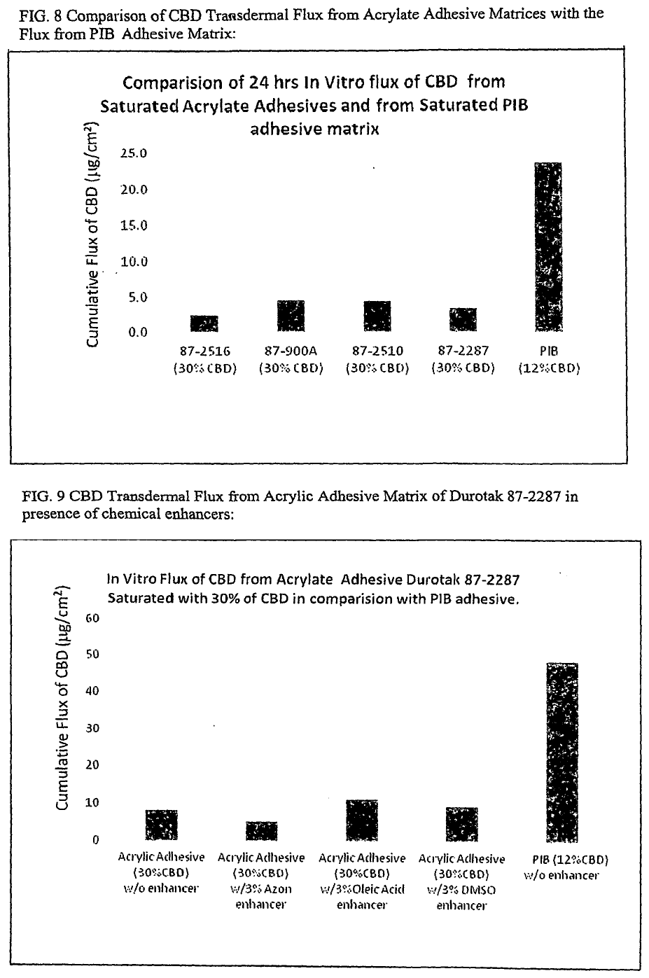

FIGS. 8 and 9 show transdermal fluxes of CBD from monolithic patches. The importance of those results are as follows. FIG. 8 shows comparison of CBD flux from monolithic patches formulated with acrylic adhesives containing 30% of CBD and PIB adhesive containing 12% CBD. It shows that even with 3 times higher concentration of CBD in acrylic adhesives the CBD flux is much lower than the CBD flux from PIB adhesive of lower concentration (12%) of CBD. Therefore the PIB formulation is superior to acrylic formulations.

FIG. 9 shows CBD flux from acrylic monolithic patches with 30% CBD and addition best enhancers at 3%. Even with addition of enhancers the CBD flux is much lower than from PIB adhesive. Therefore the PIB formulation is superior to acrylic formulations with enhancers.

Not shown, but tested by the present inventors, is that formulations with silicone adhesive BIO PSA 7-4302 showed no flux of CBD. Therefore the PIB formulation is superior to silicone adhesive monolithic formulations.

Dermal Patch Data of the Present Disclosure

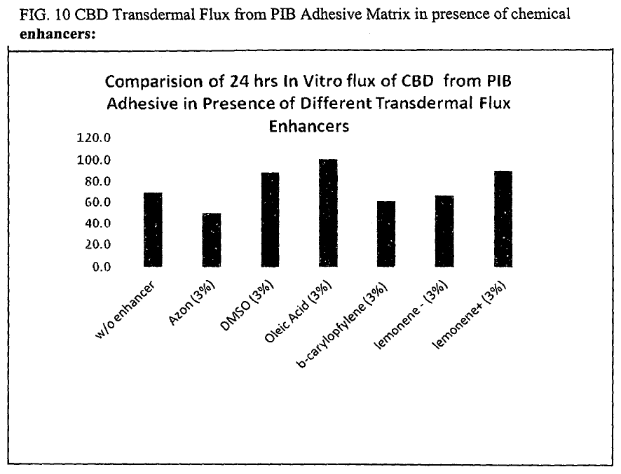

FIG. 10 shows Comparison of 24 hours of In Vitro flux of CBD from PIB Adhesive in Presence of Different Transdermal Flux Enhancers. FIG. 10 refers to a monolithic patch. Oleic Acid at 3% concentration in PIB adhesive saturated with CBD has the best enhancement effect on CBD transdermal flux increasing the flux merely by about 50%. FIG. 10 shows that the Enhancement Factor (increase in 24 hour flux) was 1.5-fold.

Transdermal flux of cannabinoids was measured In Vitro through human cadaver epidermis using Franz Diffusion Cell Method. Samples of 300 microliters were taken of the receiving solution and analyzed by HPLC for amount of cannabinoid substance that passed through the epidermis.

The results of CBD fluxes were obtained for CBD solubilized in pure liquid enhancers. Such formulations is novel in application in reservoir patch delivering greater transdermal dose of cannabinoids, providing that they do not cause the skin irritation. FIG. 10 shows results of CBD flux from PIB monolithic patch all with 12% CBD (saturation concentration of CBD in PIB adhesive) and 3% of enhancer.

In embodiments, this discloses ratios of oleic acid, DMSO, and limonene, that are suitable for monolithic patch of FIG. 10, for monolithic patches other than that represented by FIG. 10, and optionally, suitable for reservoir patches. Suitable ratios (vol/vol/vol) include, oleic acid/DMSO/limonene at ratios of about 5/5/90; about 5/10/85; about 5/15/80; about 5/20/75; about 5/25/70; about 5/30/65; about 5/35/60; about 5/40/55; about 5/45/50; about 5/50/45; about 5/55/40; about 5/60/35; about 5/65/30; about 5/70/25; about 5/75/20; about 5/80/15/about 5/85/10; and about 5/90/5, and so on.

Also, suitable ratios (vol/vol/vol) include oleic acid/DMSO/limonene at ratios of about 10/5/85; about 10/10/80; about 10/15/75; about 10/20/70; about 10/25/65; about 10/30/60; about 10/35/55/about 10/40/50; about 10/45/45/about 10/50/40; about 10/55/35; about 10/60/30; about 10/65/25; about 10/70/20; about 10/75/15/about 10/80/10; about 10/85/5; and so on.

Moreover, suitable ratios (vol/vol) include oleic acid/DMSO/limonene at ratios of about 20/5/75; about 20/10/70; about 20/15/65; about 20/20/60; about 20/25/55; about 20/30/50; about 20/35/45; about 20/40/40; about 20/45/35; about 20/50/30; about 20/55/25; about 20/60/20; about 20/65/15; about 20/70/10; about 20/75/5, and the like.

Additionally, suitable ratios (vol/vol/vol) include oleic acid/DMSO/limonene at ratios of about 40/5/55/about 40/10/50; about 40/15/45; about 40/20/40; about 40/25/35; about 40/30/30; about 40/35/25; about 40/40/20; about 40/45/15; about 40/50/10; about 40/55/5; and so on.

Further suitable ratios (vol/vol/vol) include oleic acid/DMSO/limonene at ratios of about 50/5/45; about 50/10/40; about 50/15/35; about 50/20/30; about 50/25/25; about 50/30/20; about 50/35/15; about 50/40/10; about 50/45/5; and the like.

Suitable ratios (vol/vol/vol) also include oleic acid/DMSO/limonene at ratios of about 60/5/35; about 60/10/30; about 60/15/25; about 60/20/20; about 60/25/15; about 60/30/10; about 60/35/5; and so on.

Also, suitable ratios (vol/vol/vol) include oleic acid/DMSO/limonene at ratios of about 70/5/25; about 70/10/20; about 70/15/15; about 70/20/10; about 70/25/5, and the like.

Suitable ratios (vol/vol/vol) further include oleic acid/DMSO/limonene at ratios of about 80/5/15; about 80/10/10; about 80/15/5. Other suitable ratios (vol/vol/vol) are oleic acid/DMSO/limonene at ratios of about 90/5/5.

Regarding the term “about,” in the above lists of ratios, the term about means that any given ratio encompasses the range from the next lower ratio to the next higher ratio (but not including the values that bracket this range). In exclusionary embodiments, the present disclosure can exclude any composition, and can exclude any device that contains any composition, where the composition meets the values or ranges, as set forth above. Where necessary, for exclusionary embodiments, the above ratios can be used to refer to vol/vol/vol or to wt/wt/wt.

Dermal Patch Data of the Present Disclosure

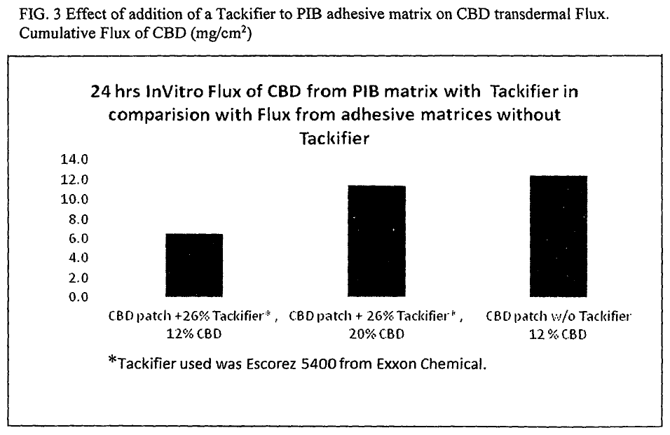

FIG. 3 shows 24 hour In Vitro Flux of CBD from PIB matrix with Tackifier in comparison with Flux from adhensive matrices without Tackifier. This concerns FIG. 3. Tackifiers are compounds that when added to adhesives, they increase tack and adhesive strength of that adhesives. Our PIB adhesive when mixed with different drugs or nutraceutical extracts loses some adhesive strength that may cause poor stickiness to skin and consequently insufficient flux of the active ingredients form the PIB adhesive to skin. Therefore, there is a need for adding a substance to the adhesive to increase its adhesive force between the adhesive and the skin. The inventor is using a PIB adhesive that provide the best partitioning of cannabinoids to skin out of all medical adhesives available acrylic type and silicone type. However, the adhesion to skin is not the best. Addition of a tackifier substance would be desirable. Cycloaliphatic hydrocarbon resins can be used to tackify a variety of adhesive polymers. However, they have not been used in PIB adhesives. We found that one of the cycloaliphatic resins manufactured by Exxon Mobil Chemical Escorez® 5400 was compatible with our PIB adhesive. At concentration of 26%, the adhesive strength of the KB matrix doubled tested on human skin. The inventor observed that addition of a Escorez 5400 tackifier to PIB adhesive increased solubility of CBD in the matrix. Therefore the inventor had to increase the concentration of CBD in the matrix to achieve the saturation of CBD and thus the optimum partitioning into the skin.

This concerns FIG. 3. The in vitro results indicate that indeed at CBD concentration of 20% the saturation was achieved, and the flux of CBD was practically the same as the CBD flux from saturated un-tackified PIB adhesive matrix contained 12% CBD at saturation concentration. The chemical composition of PIB adhesive is PIB polymer of Molecular weight between 12,000-50,000 Daltons solubilized in heptane yielding solution of about 3,000 centipoises. The tackifying resin Escorez® 5400 was dissolved in heptane 3 parts of Escorez 5400 to 1 part of heptane and added to PIB solution while mixing. Mixing was done with motorized mixer using sheering mixing blade. The term “solid matrix” means semisolid tacky matter as opposed to a liquid free flowing solution or highly thixotropic solution such as eg. ketchup.

This concerns determining percent saturation of CBD in PIB adhesive, and determining percent saturation of CBD in PIG adhesive that contains tackifier. Regarding FIG. 3, percent saturation was determined using both: (1) Testing increased concentration of CBD in the adhesive matrix until appearance of turbidity; and (2) Running flux experiment for patches formulations with increasing concentration finding the onset concentration for which the transdermal flux reached platau.

Regarding peel strength of adhesives, the inventor used judgement and estimation for preparing the patch with 26% of tackifying resin. Escorez 5400 has been used in many adhesives but not in PIB. The inventor was the first to use it, and the inventor's results demonstrated that it worked as a tackifyer. The inventor estimated the peel strength of the formulation with tackifier and without tackifier, by peeling them both from the inventor's own arm skin. To obtain saturation concentration of CBD in the PIB adhesive with 26% of tackifying resin Escorez 5400 the inventor needed to add more CBD to reach turbidity. The concentration of the saturation was about 18% CBD in PIB adhesive. The transdermal flux of CBD however did not increase. It was the same as the flux from PIB saturated with CBD without tackifier.

The benefit of using tackifier would be to use it in formulations that may contain additional active ingredients together with cannabinoids that might affect adversally adhesion of the patch to skin like addition of liquid terpenes.

The inventor found that the Escorez 5400 tackifier increased solubility of CBD in PIB adhensive, where this permits greater concentrations of CBD to be used in manufacturing dermal patch. This is expected to result in thus greater flux of CBD to a patient, where dermal patch is in contact to skin of a patient.

In embodiments, the present disclosure provides a patch with about 26% tackifying resin, that is, about 26% tackifier, about 12%, about 14%, about 16%, about 18%, about 20%, about 22%, about 24%, about 26%, about 28%, about 30%, about 32%, or about 34% tackifier, and the like. Also, the present invention provides a patch with 12%, 14%, 16%, 18%, 20%, 22%, 24%, 26%, 28%, 30%, 32%, or 34% tackifier, and the like. In range embodiments, the present disclosure provides a patch with tackifier in the range of, 10-12% tackifier, 12-14%, 14-16%, 16-18%, 18-20%, 20-22%, 22-24%, 24-26%, 26-28%, 28-30%, 30-32%, or 32-34% tackifier, and the like, or a range consisting of the sum of any two of these consecutive ranges, or the sum of any three of these consecutive ranges, or the sum of any four of these consecutive ranges, and the like. In exclusionary embodiments, the present disclosure can exclude a patch with any of the above tackifier values, with “about” tackifier values, or with any of the above tackifier ranges.

Dermal Patch Data of the Present Disclosure

Comparison of 24 Hours In Vitro Flux of CBD from Saturated Acrylate Adhesives and from Saturated PIB Adhesive Matrix (Monolithic Patches)

Comparison of CBD Transdermal Flux from Acrylate Adhesive Matrices with the Flux from PIB Adhesive Matrix. FIG. 8 shows transdermal fluxes of CBD from monolithic patches. FIG. 1 compares CBD flux from monolithic patches formulated with acrylic adhesives containing 30% of CBD and PIB adhesive containing 12% CBD. It shows that even with 3 times higher concentration of CBD in acrylic adhesives the CBD flux is much lower than the CBD flux from PIB adhesive of lower concentration (12%) of CBD. Therefore the PIB formulation is superior to acrylic formulations. Durotak 87-900A is a non-functional acrylic copolymer structured from common acrylic base monomers such as 2-Ethylhexyl Acrylate, Butyl Acrylate and Isooctyl Acrylate. Durotak 87-2510 and Durotak 87-2287 are —OH (Hydroxy) functional acrylic copolymers structured from acrylic base monomers. Hydroxy Ethyl Acrylate monomer Durotak 87-2516 is a dual functionality: —OH (Hydroxy) and —COOH (Carboxyl) structured from at least one acrylic base monomer and other two monomers such as Acrylic Acid and Hydroxy Ethyl Acrylate. Four acrylate adhesives supplied by Henkel Corporation (Stamford, Conn.; Bridgewater, N.J.) were tested for transdermal flux of CBD: Durotak 87-900A (without functional groups), Durotak 87-2516 (with both —OH and —COOH functional groups) Durotak 87-2510 and Durotak 87-2287 (both with —OH functional group). The PIB adhesive was also supplied by Henkel Corp.

High concentration of CBD is not determining factor for optimizing transdermal flux of CBD from acrylate adhesives. In vitro flux of CBD tested from silicone adhesive BIO PSA-7-4302 was undetectable. Our PIB matrix formulations provides the best thermodynamics for CBD diffusion from the matrix into skin.

### Example. Chemistry of Acrylic Adhesives and Chemistry of Tackifiers of the Present Disclosure

Duro-Tak® 87-2516 is an acrylic copolymer adhesive containing EHA, vinylacetate, and hydroxyethylacrylate. EHA is 2-ethylhexylacrylate (see, U.S. Pat. No. 5,783,208 of Venkateshwaran). Duro-Tak® 87-2516 is an acrylate-vinylacetate copolymer with a hydroxyl group (see, Zhao, Park, Kim, Lee (2002) Drug Devel. Industrial Pharmacol. 28:1125-1131). Duro-Tak® 87-2516 has viscosity of 4350 cp at 41.5% solids (see, US2006/173,124 of Paul). Duro-Tak® 87-2516 is hydroxyfunctional and crosslinked (see, US2002/0058068 of Houze). Duro-Tak® 87-2516 is an acrylate-vinyl acetate self-curing pressure-sensitive adhesive in an organic solvent (see, US2006/0121102 of Chiang).

Duro-Tak 87-4287 is a copolymer with 2-ethylhexyl acrylate as the main repeating monomer unit. Duro-Tak 87-4287 is a copolymer with vinyl acetate and contains OH-functional groups as 2-hydroxyethyl acrylate is also part of the polymer composition (Wolff (2014) Pharm. Res. 31:2186-2202).

Duro-Tak® 87-2287 is a polyacrylate adhesive. According to U.S. Pat. No. 5,693,335 of Xia, “Duro-Tak 87-2287 is a solution polyacrylate adhesive available from National Starch and Chemical Co. Its monomer composition is: vinyl acetate, 28%; 2-ethylhexyl acrylate, 67%; hydroxyethyl acrylate, 4.9% glycidal methacrylate, 0.1%. It contains no crosslinking agent. It is available as a 50% solids solution in ethyl acetate.” See also, U.S. Pat. No. 6,071,531 of Jona. According to U.S. Pat. No. 5,780,050 of Jain, Duro-Tak® 87-2287 is an acrylic adhesive free of acid functional groups. According to US2009/0258061 of Hwang, “Duro-Tak® 87-2287 is an adhesive is derived from a monomer composition of vinyl acetate, 28%; 2-ethylhexyl acrylate, 67%; hydroxyethyl acrylate, 4.9%; and glycidyl methacrylate, 0.1%, see U.S. Pat. No. 5,693,335.”

DuroTak® 87-900A is an acrylic pressure-sensitive adhesive that comprises 2-ethylhexyl acrylate, butyl acetate, t-octyl acrylamide, and methyl methacrylate. This list of chemicals was accepted, as a substitute for “DuroTak® 87-900A” by the patent examiner in file history of US2009/0297590 of Yamagi. According to a Product Selection Guide, DuroTak® 87-900A has no crosslinker, no vinyl acetate, 43% solids, viscosity of 1800 cP (see, DURO-TAK and GELVA Transdermal Pressure Sensitive Adhesives. Product Selection Guide (2013) Henkel Corp., Bridgewater, N.J. (2 pages)). According to Wolff (2014) Pharm. Res. 31:2186-2202, Dura-Tak 87-900A is, “Duro-Tak 87-900A . . . have 2-ethylhexylacrylate as the main repeating monomer unit . . . Duro-Tak 87-900A contains besides 2-ethylhexylacrylate, butylacrylate, methyl methacrylate and tertiary-octyl acrylamide units.” See also, para. 0031 of Yamagi US2009/0297590. Duro-Tak 87-900A contains 2-ethylhexyl acrylate as the main repeating monomer unit, and also contains butylacrylate, methyl methacrylate and tertiary-octyl acrylamide units (Wolff (2014) Pharm. Res. 31:2186-2202).

Duro-Tak® 87-2510 has been described as, “copolymer: acrylate; functional group: OH; 40.5% solution of noncrosslinking acrylic copolymer, 4500 cps, soluability parameter 16.” (see, Kim, Gwak, Chun (2014) Arch. Pharm. Res. 27:763-768).

Escorez® 5400 is described as, “dicyclopentadiene (DCPD) resin” (see, U.S. Pat. No. 9,296,930 of Hu); “hydrogenated polycyclopentadiene resin” (see, U.S. Pat. No. 9,039,862 of Lotz); a “hydrocarbon tackifying resin, having a molecular weight of about 400 grams/mole, a softening point of 103 degrees C., and a glass transition temperature of about 50 degrees C.” (see, U.S. Pat. No. 9,074,087 of Chen); a “cycloalphiphatic hydrocarbon tackifying resin having a ring and ball softening point from about 100 degrees C. to about 106 degrees C.” (see, U.S. Pat. No. 9,803,113 of Tse).

Escorez® 5400 has the following characteristics: softening point 218.1 degrees F., initial color: 0.6 YI; thermal color stability: 5 hours, 347 degrees F. (175 degrees C.) 6.4 YI, melt viscosity: 320 degrees F. (160 degrees C.) of 800 cP; molecular weight (number average; Mn) 400 g/mol; molecular weight (Mw) 670 g/mol; glass transition temperature (Tg): 126 degrees F. (Product Datasheet, ExxonMobil, Escorez® 5400 Tackifying Resin).

### Example. Dermal Patch Data of the Present Disclosure

In Vitro Flux of CBD from Acrylate Adhesive Durotak 87-2287 Saturated with 30% of CBD in Comparison with PIB Adhesive.

FIG. 9 shows effect of addition of enhancers to an acrylate adhesive on CBD flux. Effect of addition of an enhancer to acrylic adhesives was tested using three enhancers: Azon, CBD and Oleic Acid at 3% concentration in the Durotak 87-2287 adhesive matrix. The concentration of CBD was 30%. Addition of enhancers such as Azon, Oleic Acid and DMSO do not improve significantly the CBD flux from acrylic adhesive matrices and are still far less that CBD flux from PIB adhesive matrix even without an enhancer.

FIG. 9 shows that PIB adhesive matrix is the preferred, or optimal, choice of a monolithic adhesive matrices for high transdermal delivery of cannabinoids from monolithic patch. Azon® is a trade name for 1-Dodecylazepan-2-one. It promotes hydrophilic and water-based drugs passage into the skin. It is used in cosmetics as skin penetration enhancer.

Chemical enhancers are primarily used to increase the skin penetration passage for the active ingredients. When mixed into a matrix containing an active ingredient and placed on skin both the active ingredient and the enhancer molecules will partition into the skin. The intent of the formulation is to have the highest possibly partitioning of both: the active ingredient and the enhancer. However, one must know that the partitioning and diffusion between two phases in intimate contact are governed by thermodynamics of solubility of the diffusates in the phases being in contact.

In case of transdermal delivery systems, we visualize two phases in intimate contact. The “donor “phase that could be a monolithic adhesive, cream, gel or liquid that contain an active ingredient and an enhancer and a “receiving” phase, which is a skin. Importantly, the solubility parameters of the active ingredient substance and of the enhancer in both phases are the deciding factors of whether the active ingredient and enhancer will diffuse into the skin.

For example, since the skin's stratum corneum (the outmost layer of human skin) is lipophilic (non-polar) it provides favorable environment for solubilizing the lipophilic substance of active ingredients and enhancers. Consequently, to induce the high partitioning of the substances from the “donor” matrix, it must provide unfavorable environment for solubilizing those lipophilic substances. That means that the adhesive matrix, cream, gel or liquid must be less lipophilic than the stratum corneum or less soluble for the active ingredient substance and enhancer.

Solvent based adhesives are based on nonpolar or slightly polar polymers such as acrylic ones. Consequently, lipophilic substances will solubilize in the adhesives and poorly partitioned into the skin. Since most of enhancers are also lipophilic they also will be poorly partitioned into skin from lipophilic matrices. Therefore, most enhancers are not very productive in the monolithic patches. Enhances do not partition into skin and high rate to be effective in providing passage through skin for the active ingredients. FIG. 9 discloses the in vitro fluxes of CBD from acrylic adhesives presence of some chemical enhancers.

Advantage of the enhancers is that when mixed with drug in the transdermal system are expected to partition from the adhesive matrix into the skin. Disadvantage can be that they may cause skin irritation. Table 1 shows cumulative flux of CBD (micrograms/cm²) through human cadaver skin from 10% of CBD in penetration enhancers after 6 hours. The data show that the best penetration enhancer for transdermal delivery of CBD is oleic acid.

TABLE 1
Cumulative flux of CBD (micrograms/cm²) through human
cadaver skin from 10% of CBD in pentration enhancers,
after six hours. The data show inverse correlation of
CBD flux to its solubility in the enhancer.

| | | CBD solubility |
|---|---|---|
| Enhancer | Micrograms/cm² | (mg/mL) |
| Ethanol | 69.6 | (numbers pending) |
| 1,2-Propylene glycol | 11.9 | (numbers pending) |
| Oleic acid | 178.6 | (numbers pending) |

The inventor found that the best penetration enhancer for transdermal delivery of CBD is oleic acid. EtOH is only used as an enhancer in hydrogels. In adhesives is not used because it evaporates during drying of the adhesive matrix in the oven during manufacturing of the transdermal patches. 1,2 Propylene Glycol is used in adhesives as a mild enhancer and solubilizer of difficult to dissolve in the pure adhesive matrix drugs e.g. estradiol (Ludwig Weimann's formulation of the PMS Patch using Estradiol in an acrylic adhesive matrix) Oleic Acid has high boiling point and can be used in monolithic patch formulations. CBD patch based on PIB adhesive with 3% Oleic Acid showed only 50% increase of the CBD transdermal flux. (see above graph) EF=1.5. Enhancement Factor EF=(Transdermal Flux with enhancer/Transdermal Flux w/o enhancer). Significant enhancement is in range of EF=3-10. FIG. 9 shows CBD flux from acrylic monolithic patches with 30% CBD and addition best enhancers at 3%. Even with addition of enhancers the CBD flux is much lower than from PIE adhesive. Therefore the PIE formulation is superior to acrylic formulations with enhancers. Not shown, but tested by the inventor, was the result that formulations with silicone adhesive BIO PSA 7-4302 showed no flux of CBD. Therefore the PIB formulation is superior to silicone adhesive monolithic formulations.

### Example Comparing In Vitro Transdermal Flux of Present Disclosure with that Provided by Other Dermal Patches

Table 2 provides data comparing transdermal flux with the transdermal patch from present disclosure (LifeTech Global) with transdermal flux from transdermal patch from MarysMedicinals and from PAPA & Berkley. As shown, values for transdermal flux from present disclosure (LifeTech Global) are superior to that acquired with Franz cell tests using the comparator dermal patches:

TABLE 2
Comparison of the In Vitro Transdermal Flux of
Cannabidiol (CBD) from Different Patch Suppliers.

| | Content of | 24 hours Cumulative |
|---|---|---|
| Transdermal | Cannabidiol in | Transdermal Flux of |
| Patch | Transdermal | Cannabidiol |
| Manufacturer | Patch | (micrograms/cm²) |
| LifeTech Global | 15 mg/ | 41 |
| (Irvine, CA) | 30 cm² | |
| MarysMedicinals | 10.25mg/ | 3 |
| (Denver, CO) | 25 cm² | |
| PAPA & Barkley | 32.6 mg/ | 5 |
| (Eureka, CA) | 33 cm² | |

Comparison of the In Vitro Transdermal Flux of
Tetrahydrocannabinol (THC) from Different Patch Suppliers.

| | Content of | |
|---|---|---|
| | Tetrahydro- | 24 hour Cumulative |
| Transdermal | cannabinol in | Transdermal Flux of |
| Patch | Transdermal | Tetrahydrocannabinol |
| Manufacturer | Patch | (micrograms/cm²) |
| LifeTech Global | 12 mg/ | 19 |
| (Irvine, CA) | 30 cm² | |
| MarysMedicinals | 23 mg/ | 1 |
| (Denver, CO) | 25 cm² | |

In vitro transdermal flux testing was performed with human cadaver epidermis using Franz Diffusion Cell Method (Current Medicinal Chemistry, 2012, 19, 4671-4677).

The present invention is not to be limited by compositions, reagents, methods, diagnostics, laboratory data, and the like, of the present disclosure. Also, the present invention is not be limited by any preferred embodiments that are disclosed herein.

1. A composition capable of use in transdermal patch, wherein the composition comprises one or more of:
(a) An acrylic adhesive with non-functionality and an adhesive with only OH-functionality, further comprising one of more of enhancers selected from azone, oleic acid, and dimethylsulfoxide (DMSO);
(b) A polyisobutylene (PIB adhesive) with tackifiers that improve adhesion to skin using acrylic pressure sensitive adhesive mixed in at 1-50%, optionally with a cycloaliphatic hydrocarbon. resin;
(c) PIB adhesive with enhancers: at 3% of azone or oleic acid double the transdermal delivery from PIB. The disclosure provides a graph showing transdermal flux;
(d) Hemp oil with CBD of concentration 80-95% containing at least one terpene;
(e) semisolid hydrogel that is saturated with cannabidiol (CBD) and tetrahydroxannabinol (THC);
(f) A semisolid hydrogel comprising an oil that consists essentially of CBD and THC (80-95%, wt/vol), in combination with ethanol/water (80/20, vol/vol), optionally with one or more enhancers selected from azone, oleic acid, and limonene;
(g) A semisolid hydrogel saturated with CBD and THC (80-95%, wt/vol), wherein the oil is mixed with EtOH/water (80/20, vol/vol), optionally with one or more enhancers selected from ozone, oleic acid, and limonene; or
(h) A THC oil of THC (80-95%) mixed with 1-20% EtOH/water or with 1-10% EtOH water (80/20, vol/vol) wherein including greater than 10% of ethanol is capable of lowering flux of THC delivery as determinable with a reservoir patch.

2. A method for manufacturing the transdermal patch of claim 1, comprising the steps of combining THC, a film, an adhesive, and a backing, to generate an uncut patch, further comprising the uncut patch to produce a cut patch that is capable of applying to human skin.

3. (canceled)

4. (canceled)

5. The monolithic patch of claim 1, wherein the monolithic patch does not include any enhancers, or wherein the monolithic patch does not include any of each of these compounds: 1-dodecylazepan-2-one (azone), oleic acid, and dimethylsulfoxide (DMSO).

6. A monolithic patch comprising polyacrylate adhesive with a monomer composition that is vinyl acetate (28%); 2-ethylhexyl acrylate (67%); hydroxyethyl acrylate (4.9%); glycidal methacrylate (0.1%), wherein the polyacrylate adhesive contains no crosslinking agent and wherein the polyacrylate adhesive is free of acid functional groups (Duro-Tak® 87-2287), wherein the monolithic patch contains about 30% cannabidiol (CBD), about 3% oleic acid enhancer, and
wherein the monolithic patch is capable of delivery of at least 10 micrograms/cm2 cumulative flux of CBD, as determinable through human cadaver epidermis using a Franz diffusion cell, and
wherein the monolithic patch does not include any of polyisobutylene (PIB) adhesive, azone, dimethylsulfoxide (DMSO), ethanol or hydrogel.

7. (canceled)

8. (canceled)

9. (canceled)

10. (canceled)

11. A monolithic patch comprising polyisobutylene (PIB) adhesive, cannabidiol (CBD), and an enhancer, wherein the enhancer is oleic acid (3%) and wherein the monolithic patch is capable of mediating CBD flux of about 100 micrograms/cm², or wherein the enhancer is dimethylsulfoxide (DMSO) (3%) and wherein the monolithic patch is capable of mediating CBD flux of about 85 micrograms/cm², or wherein the enhancer is limonene (3%), and wherein the monolithic patch capable of mediating CBD flux of about 88 micrograms/cm², and
wherein in absence of enhancer the monolithic patch is capable Gf mediating CBD flux of about 70 micrograms/cm₂,
wherein flux is determinable with a human cadaver using Franz diffusion cell.

12. A reservoir patch that comprises a reservoir, and wherein the reservoir comprises cannabidiol (CBD), ethanol, and water, wherein the reservoir comprises ethano/water a ratio in the range of 65/35 (vol/vol) to 80/20 (vol/vol),
wherein the ethanol/water is saturated with CBD, and
wherein if the ethanol/water ratio is 65/35 (vol/vol), the reservoir patch is capable of delivering 24 hour cumulative flux of CBD of at least 170 micrograms/cm², and
wherein if the ethanol/water ratio is 80/20 (vol/vol), the reservoir patch is capable of delivering 24 hour cumulative flux of CBD of at least 270 micrograms/cm², and
wherein 24 hour cumulative flux id determinable by testing with human cadaver epidermis using a Franz diffusion cell.

13. A reservoir patch that comprises a reservoir, and wherein the reservoir comprises tetrahydrocannabinol (THC) oil, ethanol, and water,
wherein the reservoir comprises ethanol/water (vol/vol) at a value within a range of ratios, where the range is between 40/60 (vol/vol) to 50/20 (vol/vol), and
wherein if the ethanol/water ratio is 60/40 (vol/vol), the reservoir patch is capable delivering 24 hour THC cumulative flux of at least 25 micrograms/cm², and
wherein if the ethanol/water ratio is 80/20 (vol/vol), the reservoir patch is capable of delivering 24 hour THC cumulative flux of at least 16 micrograms/cm².
